SEQUENCE LISTING (28 sequences)

SEQ ID NO: 1 - DNA, 5095 bp, Homo sapien
gcgaaacggcagaggagccgagccccctccgcccaaggcgccctccctccgtccgcgcacaggcgccgtcgcttggaggaccaaggtgcctcccagcccgcaggggcgccgcgcgcaagcccgcgggctcttcggtggctctgccccgggactgcacctggaggcggccccggacggggatggtcagcggctgctgccgtctggctcgcgagcgggacgctgtgagggcaccatggcgctgactcccgggtgggggtcctcggcggggccggtccggccggagctctggctgctgctgtgggcagccgcgtggcgcctgggtgcctcggcgtgccccgccctctgcacctgcaccggaaccacggtggactgccacggcacggggctgcaggccattcccaagaatatacctcggaacaccgagcgcctggaactcaatggcaacaacatcactcggatccataagaatgactttgcggggctcaagcagctgcgggtgctgcagctgatggagaaccagattggagcagtggaacgtggtgcttttgatgacatgaaggagctggagcggctgcgactgaaccgaaaccagctgcacatgttaccggaactgctgttccagaacaaccaggctttgtcaagactggacttgagtgagaacgccatccaggccatccccaggaaagcttttcggggagctacggaccttaaaaatttacagctggacaagaaccagatcagctgcattgaggaaggggccttccgtgctctgcgggggctggaggtgctgaccctgaacaacaacaatatcaccaccatccccgtgtccagcttcaaccatatgcccaagctacggaccttccgcctgcactccaaccacctgttttgcgactgccacctggcctggctctcgcagtggctgaggcagcggccaaccatcgggctcttcacccagtgctcgggcccagccagcctgcgtggcctcaatgtggcagaggtccagaagagtgagttcagctgctcaggccagggagaagcggggcgcgtgcccacctgcaccctgtcctccggctcctgcccggccatgtgcacctgcagcaatggcatcgtggactgtcgtggaaaaggcctcactgccatcccggccaacctgcccgagaccatgacggagatacgcctggagctgaacggcatcaagtccatccctcctggagccttctcaccctacagaaagctacggaggatagacctgagcaacaatcagatcgctgagattgcacccgacgccttccagggcctccgctccctgaactcgctggtcctctatggaaacaagatcacagacctcccccgtggtgtgtttggaggcctatacaccctacagctcctgctcctgaatgccaacaagatcaactgcatccggcccgatgccttccaggacctgcagaacctctcactgctctccctgtatgacaacaagatccagagcctcgccaagggcactttcacctccctgcgggccatccagactctgcacctggcgcagaaccctttcatttgcgactgtaacctcaagtggctggcagacttcctgcgcaccaatcccatcgagacgagtggtgcccgctgtgccagtccccggcgcctcgccaacaagcgcatcgggcagatcaagagcaagaagttccggtgctcagccaaagagcagtacttcattccaggcacggaggattaccagctgaacagcgagtgcaacagcgacgtggtctgtccccacaagtgccgctgtgaggccaacgtggtggagtgctccagcctgaagctcaccaagatccctgagcgcatcccccagtccacggcagaactgcgattgaataacaatgagatttccatcctggaggccactgggatgtttaaaaaacttacacatctgaagaaaatcaatctgagcaacaacaaggtgtcagaaattgaagatggggccttcgagggcgcagcctctgtgagcgagctgcacctaactgccaaccagctggagtccatccggagcggcatgttccggggtctggatggcttgaggaccctaatgctgcggaacaaccgcatcagctgcatccacaacgacagcttcacgggcctgcgcaacgtccggctcctctcgctctacgacaaccagatcaccaccgtatccccaggagccttcgacaccctccagtccctctccacactgaatctcctggccaaccctttcaactgcaactgccagctggcctggctaggaggctggctacggaagcgcaagatcgtgacggggaacccgcgatgccagaaccctgactttttgcggcagattcccctgcaggacgtggccttccctgacttcaggtgtgaggaaggccaggaggaggggggctgcctgccccgcccacagtgcccacaggagtgcgcctgcctggacaccgtggtccgatgcagcaacaagcacctgcgggccctgcccaagggcattcccaagaatgtcacagaactctatttggacgggaaccagttcacgctggttccgggacagctgtctaccttcaagtacctgcagctcgtggacctgagcaacaacaagatcagttccttaagcaattcctccttcaccaacatgagccagctgaccactctgatcctcagctacaatgccctgcagtgcatcctgcctttggccttccagggactccgctccctgcgcctgctgtctctccacggcaatgacatctccaccctccaagagggcatctttgcagacgtgacctccctgtctcacctggccattggtgccaaccccctatactgtgactgccacctccgctggctgtccagctgggtgaagactggctacaaggaaccgggcattgctcgttgtgctgggccccaggacatggagggcaagctgctcctcaccacgcctgccaagaagtttgaatgccaaggtcctccaacgctggctgtccaggccaagtgtgatctctgcttgtccagtccgtgccagaaccagggcacctgccacaacgacccccttgaggtgtacaggtgcgcctgccccagcggctataagggtcgagactgtgaggtgtccctggacagctgttccagtggcccctgtgaaaatgggggcacctgccatgcacaggagggcgaggatgccccgttcacgtgctcctgtcccaccggctttgaaggaccaacctgtggggtgaacacagatgactgtgtggatcatgcctgtgccaatgggggcgtctgtgtggatggtgtgggcaactacacctgccagtgccccctgcagtatgagggaaaggcctgtgagcagctggtggacttgtgctctccggatctgaacccatgtcaacacgaggcccagtgtgtgggcaccccggatgggcccaggtgtgagtgcatgccaggttatgcaggtgacaactgcagtgagaaccaggatgactgcagggaccaccgctgccagaatggggcccagtgtatggatgaagtcaacagctactcctgcctctgtgctgagggctacagtggacagctctgtgagatccctccccatctgcctgcccccaagagcccctgtgaggggactgagtgccagaatggggccaactgtgtggaccagggcaacaggcctgtgtgccagtgcctcccaggcttcggtggccctgagtgtgagaagttgctcagtgtcaactttgtggatcgggacacttacctgcagttcactgacctgcaaaactggccacgggccaacatcacgttgcaggtctccacggcagaggacaatgggatccttctgtacaacggggacaacgaccacattgcagttgagctgtaccagggccatgtgcgtgtcagctacgacccaggcagctaccccagctctgccatctacagtgctgagacgatcaacgatgggcaattccacaccgttgagctggttgcctttgaccagatggtgaatctctccattgatggcgggagccccatgaccatggacaactttggcaaacattacacgctcaacagcgaggcgccactctatgtgggagggatgcccgtggatgtcaactcagctgccttccgcctgtggcagatcctcaacggcaccggcttccacggttgcatccgaaacctgtacatcaacaacgagctgcaggacttcaccaagacgcagatgaagccaggcgtggtgccaggctgcgaaccctgccgcaagctctactgcctgcatggcatctgccagcccaatgccaccccagggcccatgtgccactgcgaggctggctgggtgggcctgcactgtgaccagcccgctgacggcccctgccatggccacaagtgtgtccatgggcaatgcgtgcccctcgacgctctttcctacagctgccagtgccaggatgggtactcgggggcactgtgcaaccaggccggggccctggcagagccctgcagaggcctgcagtgcctgcatggccactgccaggcctcaggcaccaagggggcacactgtgtgtgtgaccccggcttttcgggcgagctgtgtgagcaagagtccgagtgccggggggaccctgtccgggactttcaccaggtccagaggggctatgccatctgccagaccacgcgccccctgtcatgggtggagtgccggggctcgtgcccaggccagggctgctgccagggccttcggctgaagcggaggaagttcacctttgagtgcagcgatgggacctcttttgccgaggaggtggaaaagcccaccaagtgtggctgtgccctctgcgcatagcgctgggcgtggacaggccggtgagggcgggcaaggggccccagccgctgcagcagcggagacagtcgccagcagctgggctggggtgcaggtcatcacaggacggctcctgggcagctgggccctcctgggtggggtggtgccagagcagccttttaaaagcaaattgcgccatagctgggggcagcgggggtgggcgaggcctgagctgcgggctgccctctccggaagtgccttgcacaaataggcgcttaataa

SEQ ID NO: 2 - DNA, 7910 bp, Homo sapien
gcgaaacggcagaggagccgagccccctccgcccaaggcgccctccctccgtccgcgcacaggcgccgtcgcttggaggaccaaggtgcctcccagcccgcaggggcgccgcgcgcaagcccgcgggctcttcggtggctctgccccgggactgcacctggaggcggccccggacggggatggtcagcggctgctgccgtctggctcgcgagcgggacgctgtgagggcaccatggcgctgactcccgggtgggggtcctcggcggggccggtccggccggagctctggctgctgctgtgggcagccgcgtggcgcctgggtgcctcggcgtgccccgccctctgcacctgcaccggaaccacggtggactgccacggcacggggctgcaggccattcccaagaatatacctcggaacaccgagcgcctggaactcaatggcaacaacatcactcggatccataagaatgactttgcggggctcaagcagctgcgggtgctgcagctgatggagaaccagattggagcagtggaacgtggtgcttttgatgacatgaaggagctggagcggctgcgactgaaccgaaaccagctgcacatgttaccggaactgctgttccagaacaaccaggctttgtcaagactggacttgagtgagaacgccatccaggccatccccaggaaagcttttcggggagctacggaccttaaaaatttacagctggacaagaaccagatcagctgcattgaggaaggggccttccgtgctctgcgggggctggaggtgctgaccctgaacaacaacaatatcaccaccatccccgtgtccagcttcaaccatatgcccaagctacggaccttccgcctgcactccaaccacctgttttgcgactgccacctggcctggctctcgcagtggctgaggcagcggccaaccatcgggctcttcacccagtgctcgggcccagccagcctgcgtggcctcaatgtggcagaggtccagaagagtgagttcagctgctcaggccagggagaagcggggcgcgtgcccacctgcaccctgtcctccggctcctgcccggccatgtgcacctgcagcaatggcatcgtggactgtcgtggaaaaggcctcactgccatcccggccaacctgcccgagaccatgacggagatacgcctggagctgaacggcatcaagtccatccctcctggagccttctcaccctacagaaagctacggaggatagacctgagcaacaatcagatcgctgagattgcacccgacgccttccagggcctccgctccctgaactcgctggtcctctatggaaacaagatcacagacctcccccgtggtgtgtttggaggcctatacaccctacagctcctgctcctgaatgccaacaagatcaactgcatccggcccgatgccttccaggacctgcagaacctctcactgctctccctgtatgacaacaagatccagagcctcgccaagggcactttcacctccctgcgggccatccagactctgcacctggcgcagaaccctttcatttgcgactgtaacctcaagtggctggcagacttcctgcgcaccaatcccatcgagacgagtggtgcccgctgtgccagtccccggcgcctcgccaacaagcgcatcgggcagatcaagagcaagaagttccggtgctcagccaaagagcagtacttcattccaggcacggaggattaccagctgaacagcgagtgcaacagcgacgtggtctgtccccacaagtgccgctgtgaggccaacgtggtggagtgctccagcctgaagctcaccaagatccctgagcgcatcccccagtccacggcagaactgcgattgaataacaatgagatttccatcctggaggccactgggatgtttaaaaaacttacacatctgaagaaaatcaatctgagcaacaacaaggtgtcagaaattgaagatggggccttcgagggcgcagcctctgtgagcgagctgcacctaactgccaaccagctggagtccatccggagcggcatgttccggggtctggatggcttgaggaccctaatgctgcggaacaaccgcatcagctgcatccacaacgacagcttcacgggcctgcgcaacgtccggctcctctcgctctacgacaaccagatcaccaccgtatccccaggagccttcgacaccctccagtccctctccacactgaatctcctggccaaccctttcaactgcaactgccagctggcctggctaggaggctggctacggaagcgcaagatcgtgacggggaacccgcgatgccagaaccctgactttttgcggcagattcccctgcaggacgtggccttccctgacttcaggtgtgaggaaggccaggaggaggggggctgcctgccccgcccacagtgcccacaggagtgcgcctgcctggacaccgtggtccgatgcagcaacaagcacctgcgggccctgcccaagggcattcccaagaatgtcacagaactctatttggacgggaaccagttcacgctggttccgggacagctgtctaccttcaagtacctgcagctcgtggacctgagcaacaacaagatcagttccttaagcaattcctccttcaccaacatgagccagctgaccactctgatcctcagctacaatgccctgcagtgcatcccgcctttggccttccagggactccgctccctgcgcctgctgtctctccacggcaatgacatctccaccctccaagagggcatctttgcagacgtgacctccctgtctcacctggccattggtgccaaccccctatactgtgactgccacctccgctggctgtccagctgggtgaagactggctacaaggaaccgggcattgctcgttgtgctgggccccaggacatggagggcaagctgctcctcaccacgcctgccaagaagtttgaatgccaaggtcctccaacgctggctgtccaggccaagtgtgatctctgcttgtccagtccgtgccagaaccagggcacctgccacaacgacccccttgaggtgtacaggtgcgcctgccccagcggctataagggtcgagactgtgaggtgtccctggacagctgttccagtggcccctgtgaaaatgggggcacctgccatgcacaggagggcgaggatgccccgttcacgtgctcctgtcccaccggctttgaaggaccaacctgtggggtgaacacagatgactgtgtggatcatgcctgtgccaatgggggcgtctgtgtggatggtgtgggcaactacacctgccagtgccccctgcagtatgagggaaaggcctgtgagcagctggtggacttgtgctctccggatctgaacccatgtcaacacgaggcccagtgtgtgggcaccccggatgggcccaggtgtgagtgcatgccaggttatgcaggtgacaactgcagtgagaaccaggatgactgcagggaccaccgctgccagaatggggcccagtgtatggatgaagtcaacagctactcctgcctctgtgctgagggctacagtggacagctctgtgagatccctccccatctgcctgcccccaagagcccctgtgaggggactgagtgccagaatggggccaactgtgtggaccagggcaacaggcctgtgtgccagtgcctcccaggcttcggtggccctgagtgtgagaagttgctcagtgtcaactttgtggatcgggacacttacctgcagttcactgacctgcaaaactggccacgggccaacatcacgttgcaggtctccacggcagaggacaatgggatccttctgtacaacggggacaacgaccacattgcagttgagctgtaccagggccatgtgcgtgtcagctacgacccaggcagctaccccagctctgccatctacagtgctgagacgatcaacgatgggcaattccacaccgttgagctggttgcctttgaccagatggtgaatctctccattgatggcgggagccccatgaccatggacaactttggcaaacattacacgctcaacagcgaggcgccactctatgtgggagggatgcccgtggatgtcaactcagctgccttccgcctgtggcagatcctcaacggcaccggcttccacggttgcatccgaaacctgtacatcaacaacgagctgcaggacttcaccaagacgcagatgaagccaggcgtggtgccaggctgcgaaccctgccgcaagctctactgcctgcatggcatctgccagcccaatgccaccccagggcccatgtgccactgcgaggctggctgggtgggcctgcactgtgaccagcccgctgacggcccctgccatggccacaagtgtgtccatgggcaatgcgtgcccctcgacgctctttcctacagctgccagtgccaggatgggtactcgggggcactgtgcaaccaggccggggccctggcagagccctgcagaggcctgcagtgcctgcatggccactgccaggcctcaggcaccaagggggcacactgtgtgtgtgaccccggcttttcgggcgagctgtgtgagcaagagtccgagtgccggggggaccctgtccgggactttcaccaggtccagaggggctatgccatctgccagaccacgcgccccctgtcatgggtggagtgccggggctcgtgcccaggccagggctgctgccagggccttcggctgaagcggaggaagttcacctttgagtgcagcgatgggacctcttttgccgaggaggtggaaaagcccaccaagtgtggctgtgccctctgcgcatagcgctgggcgtggacaggccggtgagggcgggcaaggggccccagccgctgcagcagcggagacagtcgccagcagctgggctggggtgcaggtcatcacaggacggctcctgggcagctgggccctcctgggtggggtggtgccagagcagccttttaaaagcaaattgcgccatagctgggggcagcgggggtgggcgaggcctgagctgcgggctgccctctccggaagtgccttgcacaaataggcgcttaataaatatttgttgagtgaatgtgtgcgtgaggtcaggccaagaagtgcagaacgatgacacccctccttacctgctatctgaatctggagaagaaaaatgacagccttccaaaccaacccttccctttggcctgtggcccaggctggcttggaactgggtctgtggccccagaagcctcttacccctctgcgggcaaccatgaagtactgtcagcctccccgggaagccagcctggttcattctgctgctacagaatctgctggtggtaggccaggctctggagcgggggtgccgcctcctgctggccagggagggtcggacccttgccccctgggctgactggcagctctgcagccacggcttgggaacgaggctgtgggtggaggtggttcttaggaccaggcctctgaatcctaaagttctagcatgactactgtagctgcgagggcttatgtggaggaaacagtcacaggggctgctcagggtggcagaccccactaaagagggcagagggttctttgctctagataaacaaacatcatctgcctccagacactggccacagtaggagtattggtcctgggcttccccagccaccagtcagccacaagctgtcggtgacctattggtagagggactgggtgtgagggtctgggccagggtgcttgacctgggagcagctggttcagagtccttcacaccgcaggccagtagggagcagtggaagggacagtgctccaggcattgggaagtccctgctggctctatcactcggggcaaacttctccccacctgggccttgggttcttcagctataaaatggccagaggtggggggcgggatgactaaaggaacagtgcagactcccccactgtggtcttgggaggccagaggagttagaagacctatctatctatctatctatctacattgatcacatcaaaagtatttatgtgcctaacccggggctggggattgtggacgttctggcctaatggacagatgtgaactcatcccagagcatcgcaggaatgaccaggatgcccgggaagagttgagctgagtgggggctccagccacagacagcggcccaggccagggagttgctggcaacgaaggagccagtggtggaagaagaagaggccctgaatatacgattgcctgcccacgttgtcttctcttccatacacagtgaaaatgtagaaagatggtttgtgaggccaaactgtgaatgggctaaagggaggcaaagttgcactctccttccccagagggctcaccaagagggcacacccccgggggttctggtgggcaacgggggtgagcatgtccctgccctggctccctccatctgtgaccaggaggcatggctgggtgtatgttcaggtgaggctcagagtggcattgtgtccctgtcccctgcccagggcagtgaggggagcccttgatgctgattagaaggctagaactggggtagaggtgcctggcatgtctcatgccatggggactcaatctagcaactgtgagtcctggggtccctgtgatgggaagagggcagtgccctgcccaatgtggcaggtgtcctcatggcaggatctgcccctcaccagggggctgggatctacttgcttggagctctgagcaaggccacaatgcccgcccccacccccaagtagactgcagcctgggcctcatggggcttctcccaggcccacatggcatccctctctgagtttccaggccaccgtgggaccctgcagagcatctgcaccgggctggatagggcagaaaagctcaagggcagctagcttgcctcttccctggaagaaaggtgctctgggactcaccaaccctgagaaagatagctttcctggccaccaccattccccaccaccctggagaagccaattcccaggcttgaagggcactggctggcaggaggcctcttcattctgcaggaggtggaaaggacacctgtagacaggtgatgctcacccctcacctggcgccatggggctgggaggtgagcggctggcatgtttgttcctagggagcaccatgtgagcttaaggctcccctgaccggccccaccacatggcccagcctcctagcacagcagcgctgacctcagtgcagtctgaggattggaatccaccatgagatgatgtgagagctgtgtgccccaggatcaactttttctccaacttggccatcagccagcgagttgctaaggacctgagtcagcactcacgttgcctattcacactccgcttgaaagtccggaaggtggctactgcaaaatcacccctctgagaagtcctctctccacatcttgtccccctttgtgaagacccctagttcgctctgcattttaggcatgaagagatacagcagggtgcgtccggagggagctgtggccttgcaacaccactggcaacagggccggggctcccggtgaaggtgtcaggaagtggaaaaggctggactttgtctcctctttgcctgctggtagcctaaccgcaaaagtatctctttatacagaatacttacagattctaatatatatttgtatttcattttgttacagtatttttatatgttaaagtcaacatccagcgtcttgttttgcctttcagatgctatgtggtcgtggcacgttttgttgggggtttctgtagtcgtcttgtttggatcaactcctagaggctggtttagaacaggcccatgagggagctgcacctgccctggaagtattgttttagactatgtcgatattgtctgttgtcttccatgtgaacatgacattgagtcactctg

SEQ ID NO: 3 - DNA, 5902 bp, Homo sapien
gcgaaacggcagaggagccgagccccctccgcccaaggcgccctccctccgtccgcgcacaggcgccgtcgcttggaggaccaaggtgcctcccagcccgcaggggcgccgcgcgcaagcccgcgggctcttcggtggctctgccccgggactgcacctggaggcggccccggacggggatggtcagcggctgctgccgtctggctcgcgagcgggacgctgtgagggcaccatggcgctgactcccgggtgggggtcctcggcggggccggtccggccggagctctggctgctgctgtgggcagccgcgtggcgcctgggtgcctcggcgtgccccgccctctgcacctgcaccggaaccacggtggactgccacggcacggggctgcaggccattcccaagaatatacctcggaacaccgagcgcctggaactcaatggcaacaacatcactcggatccataagaatgactttgcggggctcaagcagctgcgggtgctgcagctgatggagaaccagattggagcagtggaacgtggtgcttttgatgacatgaaggagctggagcggctgcgactgaaccgaaaccagctgcacatgttaccggaactgctgttccagaacaaccaggctttgtcaagactggacttgagtgagaacgccatccaggccatccccaggaaagcttttcggggagctacggaccttaaaaatttacagctggacaagaaccagatcagctgcattgaggaaggggccttccgtgctctgcgggggctggaggtgctgaccctgaacaacaacaatatcaccaccatccccgtgtccagcttcaaccatatgcccaagctacggaccttccgcctgcactccaaccacctgttttgcgactgccacctggcctggctctcgcagtggctgaggcagcggccaaccatcgggctcttcacccagtgctcgggcccagccagcctgcgtggcctcaatgtggcagaggtccagaagagtgagttcagctgctcaggccagggagaagcggggcgcgtgcccacctgcaccctgtcctccggctcctgcccggccatgtgcacctgcagcaatggcatcgtggactgtcgtggaaaaggcctcactgccatcccggccaacctgcccgagaccatgacggagatacgcctggagctgaacggcatcaagtccatccctcctggagccttctcaccctacagaaagctacggaggatagacctgagcaacaatcagatcgctgagattgcacccgacgccttccagggcctccgctccctgaactcgctggtcctctatggaaacaagatcacagacctcccccgtggtgtgtttggaggcctatacaccctacagctcctgctcctgaatgccaacaagatcaactgcatccggcccgatgccttccaggacctgcagaacctctcactgctctccctgtatgacaacaagatccagagcctcgccaagggcactttcacctccctgcgggccatccagactctgcacctggcgcagaaccctttcatttgcgactgtaacctcaagtggctggcagacttcctgcgcaccaatcccatcgagacgagtggtgcccgctgtgccagtccccggcgcctcgccaacaagcgcatcgggcagatcaagagcaagaagttccggtgctcagccaaagagcagtacttcattccaggcacggaggattaccagctgaacagcgagtgcaacagcgacgtggtctgtccccacaagtgccgctgtgaggccaacgtggtggagtgctccagcctgaagctcaccaagatccctgagcgcatcccccagtccacggcagaactgcgattgaataacaatgagatttccatcctggaggccactgggatgtttaaaaaacttacacatctgaagaaaatcaatctgagcaacaacaaggtgtcagaaattgaagatggggccttcgagggcgcagcctctgtgagcgagctgcacctaactgccaaccagctggagtccatccggagcggcatgttccggggtctggatggcttgaggaccctaatgctgcggaacaaccgcatcagctgcatccacaacgacagcttcacgggcctgcgcaacgtccggctcctctcgctctacgacaaccagatcaccaccgtatccccaggagccttcgacaccctccagtccctctccacactgaatctcctggccaaccctttcaactgcaactgccagctggcctggctaggaggctggctacggaagcgcaagatcgtgacggggaacccgcgatgccagaaccctgactttttgcggcagattcccctgcaggacgtggccttccctgacttcaggtgtgaggaaggccaggaggaggggggctgcctgccccgcccacagtgcccacaggagtgcgcctgcctggacaccgtggtccgatgcagcaacaagcacctgcgggccctgcccaagggcattcccaagaatgtcacagaactctatttggacgggaaccagttcacgctggttccgggacagctgtctaccttcaagtacctgcagctcgtggacctgagcaacaacaagatcagttccttaagcaattcctccttcaccaacatgagccagctgaccactctgatcctcagctacaatgccctgcagtgcatcccgcctttggccttccagggactccgctccctgcgcctgctgtctctccacggcaatgacatctccaccctccaagagggcatctttgcagacgtgacctccctgtctcacctggccattggtgccaaccccctatactgtgactgccacctccgctggctgtccagctgggtgaagactggctacaaggaaccgggcattgctcgttgtgctgggccccaggacatggagggcaagctgctcctcaccacgcctgccaagaagtttgaatgccaaggtcctccaacgctggctgtccaggccaagtgtgatctctgcttgtccagtccgtgccagaaccagggcacctgccacaacgacccccttgaggtgtacaggtgcgcctgccccagcggctataagggtcgagactgtgaggtgtccctggacagctgttccagtggcccctgtgaaaatgggggcacctgccatgcacaggagggcgaggatgccccgttcacgtgctcctgtcccaccggctttgaaggaccaacctgtggggtgaacacagatgactgtgtggatcatgcctgtgccaatgggggcgtctgtgtggatggtgtgggcaactacacctgccagtgccccctgcagtatgagggaaaggcctgtgagcagctggtggacttgtgctctccggatctgaacccatgtcaacacgaggcccagtgtgtgggcaccccggatgggcccaggtgtgagtgcatgccaggttatgcaggtgacaactgcagtgagaaccaggatgactgcagggaccaccgctgccagaatggggcccagtgtatggatgaagtcaacagctactcctgcctctgtgctgagggctacagtggacagctctgtgagatccctccccatctgcctgcccccaagagcccctgtgaggggactgagtgccagaatggggccaactgtgtggaccagggcaacaggcctgtgtgccagtgcctcccaggcttcggtggccctgagtgtgagaagttgctcagtgtcaactttgtggatcgggacacttacctgcagttcactgacctgcaaaactggccacgggccaacatcacgttgcaggtctccacggcagaggacaatgggatccttctgtacaacggggacaacgaccacattgcagttgagctgtaccagggccatgtgcgtgtcagctacgacccaggcagctaccccagctctgccatctacagtgctgagacgatcaacgatgggcaattccacaccgttgagctggttgcctttgaccagatggtgaatctctccattgatggcgggagccccatgaccatggacaactttggcaaacattacacgctcaacagcgaggcgccactctatgtgggagggatgcccgtggatgtcaactcagctgccttccgcctgtggcagatcctcaacggcaccggcttccacggttgcatccgaaacctgtacatcaacaacgagctgcaggacttcaccaagacgcagatgaagccaggcgtggtgccaggctgcgaaccctgccgcaagctctactgcctgcatggcatctgccagcccaatgccaccccagggcccatgtgccactgcgaggctggctgggtgggcctgcactgtgaccagcccgctgacggcccctgccatggccacaagtgtgtccatgggcaatgcgtgcccctcgacgctctttcctacagctgccagtgccaggatgggtactcgggggcactgtgcaaccaggccggggccctggcagagccctgcagaggcctgcagtgcctgcatggccactgccaggcctcaggcaccaagggggcacactgtgtgtgtgaccccggcttttcgggcgagctgtgtgagcaaggtcaggggcccccctcctgacgtgccctccccagggtcccccacaaattgctttagcaatttgactcttcctcccagtctcggcagcccttctgtcccttcccagccctgtaccatgggctactatggggctttcagagtccctccaccctctgggcactgctcccaatctctcctggcccagtttgccctgcagctcccttattcagggtatgcgcctcttctggctggggtctctccttgcagaagagaccacccagggtaacagctcctggcccaacctctgcctcttggccccatcacctcatgacctgggagcggcagcaggaagtccgagggttggggactccttcccaggccctccctggcctgcttgaccaagatggcttctgctggaatccggactgctaaggctgggcagggagaagctggagaaacccagaactcaggcaccttaagggtcccctaggctatcccctccccagcatgaatccttttcataatgttattgcccccggggcctccagcctctcttgcacgcctctagcaatggggaccccccctccctccagcagttcagtctgtcctggagggccctgacgctaggaccagactgtaggccctgggaggaagcttgtgcatgtcagtgggaggcggaagtatgctggggacagaggtggggtctgagagccaaaggatccaccccacagatgctgccctcctcctcctcttcctcctgcctccctggaggcagcagctcacccatgggtgtgccctgaggctttctcttggtgtctgtcccatccagagtccgagtgccggggggaccctgtccgggactttcaccaggtccagaggggctatgccatctgccagaccacgcgccccctgtcatgggtggagtgccggggctcgtgcccaggccagggctgctgccagggccttcggctgaagcggaggaagttcacctttgagtgcagcgatgggacctcttttgccgaggaggtggaaaagcccaccaagtgtggctgtgccctctgcgcatagcgctgggcgtggacaggccggtgagggcgggcaaggggccccagccgctgcagcagcggagacagtcgccagcagctgggctggggtgcaggtcatcacaggacggctcctgggcagctgggccctcctgggtggggtggtgccagagcagccttttaaaagcaaattgcgccatagctgggggcagcgggggtgggcgaggcctgagctgcgggctgccctctccggaagtgccttgcacaaataggcgcttaataa

SEQ ID NO: 4 - DNA, 8717 bp, Homo sapien
gcgaaacggcagaggagccgagccccctccgcccaaggcgccctccctccgtccgcgcacaggcgccgtcgcttggaggaccaaggtgcctcccagcccgcaggggcgccgcgcgcaagcccgcgggctcttcggtggctctgccccgggactgcacctggaggcggccccggacggggatggtcagcggctgctgccgtctggctcgcgagcgggacgctgtgagggcaccatggcgctgactcccgggtgggggtcctcggcggggccggtccggccggagctctggctgctgctgtgggcagccgcgtggcgcctgggtgcctcggcgtgccccgccctctgcacctgcaccggaaccacggtggactgccacggcacggggctgcaggccattcccaagaatatacctcggaacaccgagcgcctggaactcaatggcaacaacatcactcggatccataagaatgactttgcggggctcaagcagctgcgggtgctgcagctgatggagaaccagattggagcagtggaacgtggtgcttttgatgacatgaaggagctggagcggctgcgactgaaccgaaaccagctgcacatgttaccggaactgctgttccagaacaaccaggctttgtcaagactggacttgagtgagaacgccatccaggccatccccaggaaagcttttcggggagctacggaccttaaaaatttacagctggacaagaaccagatcagctgcattgaggaaggggccttccgtgctctgcgggggctggaggtgctgaccctgaacaacaacaatatcaccaccatccccgtgtccagcttcaaccatatgcccaagctacggaccttccgcctgcactccaaccacctgttttgcgactgccacctggcctggctctcgcagtggctgaggcagcggccaaccatcgggctcttcacccagtgctcgggcccagccagcctgcgtggcctcaatgtggcagaggtccagaagagtgagttcagctgctcaggccagggagaagcggggcgcgtgcccacctgcaccctgtcctccggctcctgcccggccatgtgcacctgcagcaatggcatcgtggactgtcgtggaaaaggcctcactgccatcccggccaacctgcccgagaccatgacggagatacgcctggagctgaacggcatcaagtccatccctcctggagccttctcaccctacagaaagctacggaggatagacctgagcaacaatcagatcgctgagattgcacccgacgccttccagggcctccgctccctgaactcgctggtcctctatggaaacaagatcacagacctcccccgtggtgtgtttggaggcctatacaccctacagctcctgctcctgaatgccaacaagatcaactgcatccggcccgatgccttccaggacctgcagaacctctcactgctctccctgtatgacaacaagatccagagcctcgccaagggcactttcacctccctgcgggccatccagactctgcacctggcgcagaaccctttcatttgcgactgtaacctcaagtggctggcagacttcctgcgcaccaatcccatcgagacgagtggtgcccgctgtgccagtccccggcgcctcgccaacaagcgcatcgggcagatcaagagcaagaagttccggtgctcagccaaagagcagtacttcattccaggcacggaggattaccagctgaacagcgagtgcaacagcgacgtggtctgtccccacaagtgccgctgtgaggccaacgtggtggagtgctccagcctgaagctcaccaagatccctgagcgcatcccccagtccacggcagaactgcgattgaataacaatgagatttccatcctggaggccactgggatgtttaaaaaacttacacatctgaagaaaatcaatctgagcaacaacaaggtgtcagaaattgaagatggggccttcgagggcgcagcctctgtgagcgagctgcacctaactgccaaccagctggagtccatccggagcggcatgttccggggtctggatggcttgaggaccctaatgctgcggaacaaccgcatcagctgcatccacaacgacagcttcacgggcctgcgcaacgtccggctcctctcgctctacgacaaccagatcaccaccgtatccccaggagccttcgacaccctccagtccctctccacactgaatctcctggccaaccctttcaactgcaactgccagctggcctggctaggaggctggctacggaagcgcaagatcgtgacggggaacccgcgatgccagaaccctgactttttgcggcagattcccctgcaggacgtggccttccctgacttcaggtgtgaggaaggccaggaggaggggggctgcctgccccgcccacagtgcccacaggagtgcgcctgcctggacaccgtggtccgatgcagcaacaagcacctgcgggccctgcccaagggcattcccaagaatgtcacagaactctatttggacgggaaccagttcacgctggttccgggacagctgtctaccttcaagtacctgcagctcgtggacctgagcaacaacaagatcagttccttaagcaattcctccttcaccaacatgagccagctgaccactctgatcctcagctacaatgccctgcagtgcatcccgcctttggccttccagggactccgctccctgcgcctgctgtctctccacggcaatgacatctccaccctccaagagggcatctttgcagacgtgacctccctgtctcacctggccattggtgccaaccccctatactgtgactgccacctccgctggctgtccagctgggtgaagactggctacaaggaaccgggcattgctcgttgtgctgggccccaggacatggagggcaagctgctcctcaccacgcctgccaagaagtttgaatgccaaggtcctccaacgctggctgtccaggccaagtgtgatctctgcttgtccagtccgtgccagaaccagggcacctgccacaacgacccccttgaggtgtacaggtgcgcctgccccagcggctataagggtcgagactgtgaggtgtccctggacagctgttccagtggcccctgtgaaaatgggggcacctgccatgcacaggagggcgaggatgccccgttcacgtgctcctgtcccaccggctttgaaggaccaacctgtggggtgaacacagatgactgtgtggatcatgcctgtgccaatgggggcgtctgtgtggatggtgtgggcaactacacctgccagtgccccctgcagtatgagggaaaggcctgtgagcagctggtggacttgtgctctccggatctgaacccatgtcaacacgaggcccagtgtgtgggcaccccggatgggcccaggtgtgagtgcatgccaggttatgcaggtgacaactgcagtgagaaccaggatgactgcagggaccaccgctgccagaatggggcccagtgtatggatgaagtcaacagctactcctgcctctgtgctgagggctacagtggacagctctgtgagatccctccccatctgcctgcccccaagagcccctgtgaggggactgagtgccagaatggggccaactgtgtggaccagggcaacaggcctgtgtgccagtgcctcccaggcttcggtggccctgagtgtgagaagttgctcagtgtcaactttgtggatcgggacacttacctgcagttcactgacctgcaaaactggccacgggccaacatcacgttgcaggtctccacggcagaggacaatgggatccttctgtacaacggggacaacgaccacattgcagttgagctgtaccagggccatgtgcgtgtcagctacgacccaggcagctaccccagctctgccatctacagtgctgagacgatcaacgatgggcaattccacaccgttgagctggttgcctttgaccagatggtgaatctctccattgatggcgggagccccatgaccatggacaactttggcaaacattacacgctcaacagcgaggcgccactctatgtgggagggatgcccgtggatgtcaactcagctgccttccgcctgtggcagatcctcaacggcaccggcttccacggttgcatccgaaacctgtacatcaacaacgagctgcaggacttcaccaagacgcagatgaagccaggcgtggtgccaggctgcgaaccctgccgcaagctctactgcctgcatggcatctgccagcccaatgccaccccagggcccatgtgccactgcgaggctggctgggtgggcctgcactgtgaccagcccgctgacggcccctgccatggccacaagtgtgtccatgggcaatgcgtgcccctcgacgctctttcctacagctgccagtgccaggatgggtactcgggggcactgtgcaaccaggccggggccctggcagagccctgcagaggcctgcagtgcctgcatggccactgccaggcctcaggcaccaagggggcacactgtgtgtgtgaccccggcttttcgggcgagctgtgtgagcaaggtcaggggcccccctcctgacgtgccctccccagggtcccccacaaattgctttagcaatttgactcttcctcccagtctcggcagcccttctgtcccttcccagccctgtaccatgggctactatggggctttcagagtccctccaccctctgggcactgctcccaatctctcctggcccagtttgccctgcagctcccttattcagggtatgcgcctcttctggctggggtctctccttgcagaagagaccacccagggtaacagctcctggcccaacctctgcctcttggccccatcacctcatgacctgggagcggcagcaggaagtccgagggttggggactccttcccaggccctccctggcctgcttgaccaagatggcttctgctggaatccggactgctaaggctgggcagggagaagctggagaaacccagaactcaggcaccttaagggtcccctaggctatcccctccccagcatgaatccttttcataatgttattgcccccggggcctccagcctctcttgcacgcctctagcaatggggaccccccctccctccagcagttcagtctgtcctggagggccctgacgctaggaccagactgtaggccctgggaggaagcttgtgcatgtcagtgggaggcggaagtatgctggggacagaggtggggtctgagagccaaaggatccaccccacagatgctgccctcctcctcctcttcctcctgcctccctggaggcagcagctcacccatgggtgtgccctgaggctttctcttggtgtctgtcccatccagagtccgagtgccggggggaccctgtccgggactttcaccaggtccagaggggctatgccatctgccagaccacgcgccccctgtcatgggtggagtgccggggctcgtgcccaggccagggctgctgccagggccttcggctgaagcggaggaagttcacctttgagtgcagcgatgggacctcttttgccgaggaggtggaaaagcccaccaagtgtggctgtgccctctgcgcatagcgctgggcgtggacaggccggtgagggcgggcaaggggccccagccgctgcagcagcggagacagtcgccagcagctgggctggggtgcaggtcatcacaggacggctcctgggcagctgggccctcctgggtggggtggtgccagagcagccttttaaaagcaaattgcgccatagctgggggcagcgggggtgggcgaggcctgagctgcgggctgccctctccggaagtgccttgcacaaataggcgcttaataaatatttgttgagtgaatgtgtgcgtgaggtcaggccaagaagtgcagaacgatgacacccctccttacctgctatctgaatctggagaagaaaaatgacagccttccaaaccaacccttccctttggcctgtggcccaggctggcttggaactgggtctgtggccccagaagcctcttacccctctgcgggcaaccatgaagtactgtcagcctccccgggaagccagcctggttcattctgctgctacagaatctgctggtggtaggccaggctctggagcgggggtgccgcctcctgctggccagggagggtcggacccttgccccctgggctgactggcagctctgcagccacggcttgggaacgaggctgtgggtggaggtggttcttaggaccaggcctctgaatcctaaagttctagcatgactactgtagctgcgagggcttatgtggaggaaacagtcacaggggctgctcagggtggcagaccccactaaagagggcagagggttctttgctctagataaacaaacatcatctgcctccagacactggccacagtaggagtattggtcctgggcttccccagccaccagtcagccacaagctgtcggtgacctattggtagagggactgggtgtgagggtctgggccagggtgcttgacctgggagcagctggttcagagtccttcacaccgcaggccagtagggagcagtggaagggacagtgctccaggcattgggaagtccctgctggctctatcactcggggcaaacttctccccacctgggccttgggttcttcagctataaaatggccagaggtggggggcgggatgactaaaggaacagtgcagactcccccactgtggtcttgggaggccagaggagttagaagacctatctatctatctatctatctacattgatcacatcaaaagtatttatgtgcctaacccggggctggggattgtggacgttctggcctaatggacagatgtgaactcatcccagagcatcgcaggaatgaccaggatgcccgggaagagttgagctgagtgggggctccagccacagacagcggcccaggccagggagttgctggcaacgaaggagccagtggtggaagaagaagaggccctgaatatacgattgcctgcccacgttgtcttctcttccatacacagtgaaaatgtagaaagatggtttgtgaggccaaactgtgaatgggctaaagggaggcaaagttgcactctccttccccagagggctcaccaagagggcacacccccgggggttctggtgggcaacgggggtgagcatgtccctgccctggctccctccatctgtgaccaggaggcatggctgggtgtatgttcaggtgaggctcagagtggcattgtgtccctgtcccctgcccagggcagtgaggggagcccttgatgctgattagaaggctagaactggggtagaggtgcctggcatgtctcatgccatggggactcaatctagcaactgtgagtcctggggtccctgtgatgggaagagggcagtgccctgcccaatgtggcaggtgtcctcatggcaggatctgcccctcaccagggggctgggatctacttgcttggagctctgagcaaggccacaatgcccgcccccacccccaagtagactgcagcctgggcctcatggggcttctcccaggcccacatggcatccctctctgagtttccaggccaccgtgggaccctgcagagcatctgcaccgggctggatagggcagaaaagctcaagggcagctagcttgcctcttccctggaagaaaggtgctctgggactcaccaaccctgagaaagatagctttcctggccaccaccattccccaccaccctggagaagccaattcccaggcttgaagggcactggctggcaggaggcctcttcattctgcaggaggtggaaaggacacctgtagacaggtgatgctcacccctcacctggcgccatggggctgggaggtgagcggctggcatgtttgttcctagggagcaccatgtgagcttaaggctcccctgaccggccccaccacatggcccagcctcctagcacagcagcgctgacctcagtgcagtctgaggattggaatccaccatgagatgatgtgagagctgtgtgccccaggatcaactttttctccaacttggccatcagccagcgagttgctaaggacctgagtcagcactcacgttgcctattcacactccgcttgaaagtccggaaggtggctactgcaaaatcacccctctgagaagtcctctctccacatcttgtccccctttgtgaagacccctagttcgctctgcattttaggcatgaagagatacagcagggtgcgtccggagggagctgtggccttgcaacaccactggcaacagggccggggctcccggtgaaggtgtcaggaagtggaaaaggctggactttgtctcctctttgcctgctggtagcctaaccgcaaaagtatctctttatacagaatacttacagattctaatatatatttgtatttcattttgttacagtatttttatatgttaaagtcaacatccagcgtcttgttttgcctttcagatgctatgtggtcgtggcacgttttgttgggggtttctgtagtcgtcttgtttggatcaactcctagaggctggtttagaacaggcccatgagggagctgcacctgccctggaagtattgttttagactatgtcgatattgtctgttgtcttccatgtgaacatgacattgagtcactctg

SEQ ID NO: 5 - DNA, 767 bp, Homo sapien
  misc_feature (762)..(762): n is a, c, g, or t
gcgaaacggcagaggagccgagccccctccgcccaaggcgccctccctccgtccgcgcacaggcgccgtcgcttggaggaccaaggtgcctcccagcccgcaggggcgccgcgcgcaagcccgcgggctcttcggtggctctgccccgggactgcacctggaggcggccccggacggggatggtcagcggctgctgccgtctggctcgcgagcgggacgctgtgagggcaccatggcgctgactcccgggtgggggtcctcggcggggccggtccggccggagctctggctgctgctgtgggcagccgcgtggcgcctgggtgcctcggcgtgccccgccctctgcacctgcaccggaaccacggtggactgccacggcacggggctgcaggccattcccaagaatatacctcggaacaccgagcgcctggaactcaatggcaacaacatcactcggatccataagaatgactttgcggggctcaagcagctgcgggtgctgcagctgatggagaaccagattggagcagtggaacgtggtgcttttgatgacatgaaggagctggagcggctgcgactgaaccgaaaccagctgcacatgttaccggaactgctgttccagaacaaccaggctttgtcaagactgatctatggctcagagaggtgatttgccttggaccacagagcagcttgacaatagggttggaattagacaccaggccttctaggggccctgaagagcctgaaaatggtcttaaaccntgacc

SEQ ID NO: 6 - protein, 1534 aa, Homo sapien
MALTPGWGSSAGPVRPELWLLLWAAAWRLGASACPALCTCTGTTVDCHGTGLQAIPKNIPRNTERLELNGNNITRIHKNDFAGLKQLRVLQLMENQIGAVERGAFDDMKELERLRLNRNQLHMLPELLFQNNQALSRLDLSENAIQAIPRKAFRGATDLKNLRLDKNQISCIEEGAFRALRGLEVLTLNNNNITTIPVSSFNHMPKLRTFRLHSNHLFCDCHLAWLSQWLRQRPTIGLFTQCSGPASLRGLNVAEVQKSEFSCSGQGEAGRVPTCTLSSGSCPAMCTCSNGIVDCRGKGLTAIPANLPETMTEIRLELNGIKSIPPGAFSPYRKLRRIDLSNNQIAEIAPDAFQGLRSLNSLVLYGNKITDLPRGVFGGLYTLQLLLLNANKINCIRPDAFQDLQNLSLLSLYDNKIQSLAKGTFTSLRAIQTLHLAQNPFICDCNLKWLADFLRTNPIETSGARCASPRRLANKRIGQIKSKKFRCSAKEQYFIPGTEDYQLNSECNSDVVCPHKCRCEANVVECSSLKLTKIPERIPQSTAELRLNNNEISILEATGMFKKLTHLKKINLSNNKVSEIEDGAFEGAASVSELHLTANQLESIRSGMFRGLDGLRTLMLRNNRISCIHNDSFTGLRNVRLLSLYDNQITTVSPGAFDTLQSLSTLNLLANPFNCNCQLAWLGGWLRKRKIVTGNPRCQNPDFLRQIPLQDVAFPDFRCEEGQEEGGCLPRPQCPQECACLDTVVRCSNKHLRALPKGIPKNVTELYLDGNQFTLVPGQLSTFKYLQLVDLSNNKISSLSNSSFTNMSQLTTLILSYNALQCIPPLAFQGLRSLRLLSLHGNDISTLQEGIFADVTSLSHLAIGANPLYCDCHLRWLSSWVKTGYKEPGIARCAGPQDMEGKLLLTTPAKKFECQGPPTLAVQAKCDLCLSSPCQNQGTCHNDPLEVYRCACPSGYKGRDCEVSLNSCSSGPCENGGTCHAQEGEDAPFTCSCPTGFEGPTCGVNTDDCVDHACANGGVCVDGVGNYTCQCPLQYEGKACEQLVDLCSPDLNPCQHEAQCVGTPDGPRCECMPGYAGDNCSENQDDCRDHRCQNGAQCMDEVNSYSCLCAEGYSGQLCEIPPHLPAPKSPCEGTECQNGANCVDQGNRPVCQCLPGFGGPECEKLLSVNFVDRDTYLQFTDLQNWPRANITLQVSTAEDNGILLYNGDNDHIAVELYQGHVRVSYDPGSYPSSAIYSAETINDGQFHTVELVAFDQMVNLSIDGGSPMTMDNFGKHYTLNSEAPLYVGGMPVDVNSAAFRLWQILNGTGFHGCIRNLYINNELQDFTKTQMKPGVVPGCEPCRKLYCLHGICQPNATPGPMCHCEAGWVGLHCDQPADGPCHGHKCVHGQCVPLDALSYSCQCQDGYSGALCNQAGALAEPCRGLQCLHGHCQASGTKGAHCVCDPGFSGELCEQESECRGDPVRDFHQVQRGYAICQTTRPLSWVECRGSCPGQGCCQGLRLKRRKFTFECSDGTSFAEEVEKPTKCGCALCA

SEQ ID NO: 7 - protein, 1461 aa, Homo sapien
MALTPGWGSSAGPVRPELWLLLWAAAWRLGASACPALCTCTGTTVDCHGTGLQAIPKNIPRNTERLELNGNNITRIHKNDFAGLKQLRVLQLMENQIGAVERGAFDDMKELERLRLNRNQLHMLPELLFQNNQALSRLDLSENAIQAIPRKAFRGATDLKNLRLDKNQISCIEEGAFRALRGLEVLTLNNNNITTIPVSSFNHMPKLRTFRLHSNHLFCDCHLAWLSQWLRQRPTIGLFTQCSGPASLRGLNVAEVQKSEFSCSGQGEAGRVPTCTLSSGSCPAMCTCSNGIVDCRGKGLTAIPANLPETMTEIRLELNGIKSIPPGAFSPYRKLRRIDLSNNQIAEIAPDAFQGLRSLNSLVLYGNKITDLPRGVFGGLYTLQLLLLNANKINCIRPDAFQDLQNLSLLSLYDNKIQSLAKGTFTSLRAIQTLHLAQNPFICDCNLKWLADFLRTNPIETSGARCASPRRLANKRIGQIKSKKFRCSAKEQYFIPGTEDYQLNSECNSDVVCPHKCRCEANVVECSSLKLTKIPERIPQSTAELRLNNNEISILEATGMFKKLTHLKKINLSNNKVSEIEDGAFEGAASVSELHLTANQLESIRSGMFRGLDGLRTLMLRNNRISCIHNDSFTGLRNVRLLSLYDNQITTVSPGAFDTLQSLSTLNLLANPFNCNCQLAWLGGWLRKRKIVTGNPRCQNPDFLRQIPLQDVAFPDFRCEEGQEEGGCLPRPQCPQECACLDTVVRCSNKHLRALPKGIPKNVTELYLDGNQFTLVPGQLSTFKYLQLVDLSNNKISSLSNSSFTNMSQLTTLILSYNALQCIPPLAFQGLRSLRLLSLHGNDISTLQEGIFADVTSLSHLAIGANPLYCDCHLRWLSSWVKTGYKEPGIARCAGPQDMEGKLLLTTPAKKFECQGPPTLAVQAKCDLCLSSPCQNQGTCHNDPLEVYRCACPSGYKGRDCEVSLNSCSSGPCENGGTCHAQEGEDAPFTCSCPTGFEGPTCGVNTDDCVDHACANGGVCVDGVGNYTCQCPLQYEGKACEQLVDLCSPDLNPCQHEAQCVGTPDGPRCECMPGYAGDNCSENQDDCRDHRCQNGAQCMDEVNSYSCLCAEGYSGQLCEIPPHLPAPKSPCEGTECQNGANCVDQGNRPVCQCLPGFGGPECEKLLSVNFVDRDTYLQFTDLQNWPRANITLQVSTAEDNGILLYNGDNDHIAVELYQGHVRVSYDPGSYPSSAIYSAETINDGQFHTVELVAFDQMVNLSIDGGSPMTMDNFGKHYTLNSEAPLYVGGMPVDVNSAAFRLWQILNGTGFHGCIRNLYINNELQDFTKTQMKPGVVPGCEPCRKLYCLHGICQPNATPGPMCHCEAGWVGLHCDQPADGPCHGHKCVHGQCVPLDALSYSCQCQDGYSGALCNQAGALAEPCRGLQCLHGHCQASGTKGAHCVCDPGFSGELCEQGQGPPS

SEQ ID NO: 8 - protein, 144 aa, Homo sapien
MALTPGWGSSAGPVRPELWLLLWAAAWRLGASACPALCTCTGTTVDCHGTGLQAIPKNIPRNTERLELNGNNITRIHKNDFAGLKQLRVLQLMENQIGAVERGAFDDMKELERLRLNRNQLHMLPELLFQNNQALSRLIYGSER

SEQ ID NO: 9 - protein, 16 aa, Homo sapien
CSDGTSFAEEVEKPTK

SEQ ID NO: 10 - protein, 16 aa, Homo sapien
QYFIPGTEDYQLNSEC

SEQ ID NO: 11 - protein, 15 aa, Homo sapien
QESECRGDPVRDFHE

SEQ ID NO: 12 - protein, 14 aa, Homo sapien
EEGQEEGGCLPRQC

SEQ ID NO: 13 - protein, 15 aa, Homo sapien
NNELQDFTKTQMKPG

SEQ ID NO: 14 - protein, 8 aa, Homo sapien
CEQGQPPS

SEQ ID NO: 15 - RNA, 22 nt, Artificial
  Novel Sequence
ggggaugguggugauauuguug

SEQ ID NO: 16 - RNA, 21 nt, Artificial
  Novel Sequence
agauggggagggaucucacag

SEQ ID NO: 17 - RNA, 21 nt, Artificial
  Novel Sequence
ugaacuuccuccgcuucagcc

SEQ ID NO: 18 - RNA, 22 nt, Artificial
  Novel Sequence
uuucuccagcuucucccugccc

SEQ ID NO: 19 - RNA, 22 nt, Artificial
  Novel Sequence
cuauugucaagcugcucugugg

SEQ ID NO: 20 - RNA, 26 nt, Artificial
  Novel Sequence
guuccugaugagucgacacgaaacug

SEQ ID NO: 21 - RNA, 26 nt, Artificial
  Novel Sequence
caggcugaugagucgacacgaaagau

SEQ ID NO: 22 - RNA, 26 nt, Artificial
  Novel Sequence
ccggcugaugagucgacacgaaacuc

SEQ ID NO: 23 - RNA, 26 nt, Artificial
  Novel Sequence
gauucugaugagucgacacgaaaugc

SEQ ID NO: 24 - RNA, 26 nt, Artificial
  Novel Sequence
ucuucugaugagucgacacgaaaggg

SEQ ID NO: 25 - DNA, 3386 bp, Homo sapiens
gagggcaccatggcgctgactcccgggtgggggtcctcggcggggccggtccggccggagctctggctgctgctgtgggcagccgcgtggcgcctgggtgcctcggcgtgccccgccctctgcacctgcaccggaaccacggtggactgccacggcacggggctgcaggccattcccaagaatatacctcggaacaccgagcgcctggaactcaatggcaacaacatcactcggatccataagaatgactttgcggggctcaagcagctgcgggtgctgcagctgatggagaaccagattggagcagtggaacgtggtgcttttgatgacatgaaggagctggagcggctgcgactgaaccgaaaccagctgcacatgttaccggaactgctgttccagaacaaccaggctttgtcaagactggacttgagtgagaacgccatccaggccatccccaggaaagcttttcggggagctacggaccttaaaaatttacagctggacaagaaccagatcagctgcattgaggaaggggccttccgtgctctgcgggggctggaggtgctgaccctgaacaacaacaatatcaccaccatccccgtgtccagcttcaaccatatgcccaagctacggaccttccgcctgcactccaaccacctgttttgcgactgccacctggcctggctctcgcagtggctgaggcagcggccaaccatcgggctcttcacccagtgctcgggcccagccagcctgcgtggcctcaatgtggcagaggtccagaagagtgagttcagctgctcaggccagggagaagcggggcgcgtgcccacctgcaccctgtcctccggctcctgcccggccatgtgcacctgcagcaatggcatcgtggactgtcgtggaaaaggcctcactgccatcccggccaacctgcccgagaccatgacggagatacgcctggagctgaacggcatcaagtccatccctcctggagccttctcaccctacagaaagctacggaggatagacctgagcaacaatcagatcgctgagattgcacccgacgccttccagggcctccgctccctgaactcgctggtcctctatggaaacaagatcacagacctcccccgtggtgtgtttggaggcctatacaccctacagctcctgctcctgaatgccaacaagatcaactgcatccggcccgatgccttccaggacctgcagaacctctcactgctctccctgtatgacaacaagatccagagcctcgccaagggcactttcacctccctgcgggccatccagactctgcacctggcgcagaaccctttcatttgcgactgtaacctcaagtggctggcagacttcctgcgcaccaatcccatcgagacgagtggtgcccgctgtgccagtccccggcgcctcgccaacaagcgcatcgggcagatcaagagcaagaagttccggtgctcagccaaagagcagtacttcattccaggcacggaggattaccagctgaacagcgagtgcaacagcgacgtggtctgtccccacaagtgccgctgtgaggccaacgtggtggagtgctccagcctgaagctcaccaagatccctgagcgcatcccccagtccacggcagaactgcgattgaataacaatgagatttccatcctggaggccactgggatgtttaaaaaacttacacatctgaagaaaatcaatctgagcaacaacaaggtgtcagaaattgaagatggggccttcgagggcgcagcctctgtgagcgagctgcacctaactgccaaccagctggagtccatccggagcggcatgttccggggtctggatggcttgaggaccctaatgctgcggaacaaccgcatcagctgcatccacaacgacagcttcacgggcctgcgcaacgtccggctcctctcgctctacgacaaccagatcaccaccgtatccccaggagccttcgacaccctccagtccctctccacactgaatctcctggccaaccctttcaactgcaactgccagctggcctggctaggaggctggctacggaagcgcaagatcgtgacggggaacccgcgatgccagaaccctgactttttgcggcagattcccctgcaggacgtggccttccctgacttcaggtgtgaggaaggccaggaggaggggggctgcctgccccgcccacagtgcccacaggagtgcgcctgcctggacaccgtggtccgatgcagcaacaagcacctgcgggccctgcccaagggcattcccaagaatgtcacagaactctatttggacgggaaccagttcacgctggttccgggacagctgtctaccttcaagtacctgcagctcgtggacctgagcaacaacaagatcagttccttaagcaattcctccttcaccaacatgagccagctgaccactctgatcctcagctacaatgccctgcagtgcatcctgcctttggccttccagggactccgctccctgcgcctgctgtctctccacggcaatgacatctccaccctccaagagggcatctttgcagacgtgacctccctgtctcacctggccattggtgccaaccccctatactgtgactgccacctccgctggctgtccagctgggtgaagactggctacaaggaaccgggcattgctcgttgtgctgggccccaggacatggagggcaagctgctcctcaccacgcctgccaagaagtttgaatgccaaggtcctccaacgctggctgtccaggccaagtgtgatctctgcttgtccagtccgtgccagaaccagggcacctgccacaacgacccccttgaggtgtacaggtgcgcctgccccagcggctataagggtcgagactgtgaggtgtccctggacagctgttccagtggcccctgtgaaaatgggggcacctgccatgcacaggagggcgaggatgccccgttcacgtgctcctgtcccaccggctttgaaggaccaacctgtggggtgaacacagatgactgtgtggatcatgcctgtgccaatgggggcgtctgtgtggatggtgtgggcaactacacctgccagtgccccctgcagtatgagggaaaggcctgtgagcagctggtggacttgtgctctccggatctgaacccatgtcaacacgaggcccagtgtgtgggcaccccggatgggcccaggtgtgagtgcatgccaggttatgcaggtgacaactgcagtgagaaccaggatgactgcagggaccaccgctgccagaatggggcccagtgtatggatgaagtcaacagctactcctgcctctgtgctgagggctacagtggacagctctgtgagatccctccctaattaggtga

SEQ ID NO: 26 - DNA, 2804 bp, Homo sapiens
gagggcaccatggcgctgactcccgggtgggggtcctcggcggggccggtccggccggagctctggctgctgctgtgggcagccgcgtggcgcctgggtgcctcggcgtgccccgccctctgcacctgcaccggaaccacggtggactgccacggcacggggctgcaggccattcccaagaatatacctcggaacaccgagcgcctggaactcaatggcaacaacatcactcggatccataagaatgactttgcggggctcaagcagctgcgggtgctgcagctgatggagaaccagattggagcagtggaacgtggtgcttttgatgacatgaaggagctggagcggctgcgactgaaccgaaaccagctgcacatgttaccggaactgctgttccagaacaaccaggctttgtcaagactggacttgagtgagaacgccatccaggccatccccaggaaagcttttcggggagctacggaccttaaaaatttacagctggacaagaaccagatcagctgcattgaggaaggggccttccgtgctctgcgggggctggaggtgctgaccctgaacaacaacaatatcaccaccatccccgtgtccagcttcaaccatatgcccaagctacggaccttccgcctgcactccaaccacctgttttgcgactgccacctggcctggctctcgcagtggctgaggcagcggccaaccatcgggctcttcacccagtgctcgggcccagccagcctgcgtggcctcaatgtggcagaggtccagaagagtgagttcagctgctcaggccagggagaagcggggcgcgtgcccacctgcaccctgtcctccggctcctgcccggccatgtgcacctgcagcaatggcatcgtggactgtcgtggaaaaggcctcactgccatcccggccaacctgcccgagaccatgacggagatacgcctggagctgaacggcatcaagtccatccctcctggagccttctcaccctacagaaagctacggaggatagacctgagcaacaatcagatcgctgagattgcacccgacgccttccagggcctccgctccctgaactcgctggtcctctatggaaacaagatcacagacctcccccgtggtgtgtttggaggcctatacaccctacagctcctgctcctgaatgccaacaagatcaactgcatccggcccgatgccttccaggacctgcagaacctctcactgctctccctgtatgacaacaagatccagagcctcgccaagggcactttcacctccctgcgggccatccagactctgcacctggcgcagaaccctttcatttgcgactgtaacctcaagtggctggcagacttcctgcgcaccaatcccatcgagacgagtggtgcccgctgtgccagtccccggcgcctcgccaacaagcgcatcgggcagatcaagagcaagaagttccggtgctcagccaaagagcagtacttcattccaggcacggaggattaccagctgaacagcgagtgcaacagcgacgtggtctgtccccacaagtgccgctgtgaggccaacgtggtggagtgctccagcctgaagctcaccaagatccctgagcgcatcccccagtccacggcagaactgcgattgaataacaatgagatttccatcctggaggccactgggatgtttaaaaaacttacacatctgaagaaaatcaatctgagcaacaacaaggtgtcagaaattgaagatggggccttcgagggcgcagcctctgtgagcgagctgcacctaactgccaaccagctggagtccatccggagcggcatgttccggggtctggatggcttgaggaccctaatgctgcggaacaaccgcatcagctgcatccacaacgacagcttcacgggcctgcgcaacgtccggctcctctcgctctacgacaaccagatcaccaccgtatccccaggagccttcgacaccctccagtccctctccacactgaatctcctggccaaccctttcaactgcaactgccagctggcctggctaggaggctggctacggaagcgcaagatcgtgacggggaacccgcgatgccagaaccctgactttttgcggcagattcccctgcaggacgtggccttccctgacttcaggtgtgaggaaggccaggaggaggggggctgcctgccccgcccacagtgcccacaggagtgcgcctgcctggacaccgtggtccgatgcagcaacaagcacctgcgggccctgcccaagggcattcccaagaatgtcacagaactctatttggacgggaaccagttcacgctggttccgggacagctgtctaccttcaagtacctgcagctcgtggacctgagcaacaacaagatcagttccttaagcaattcctccttcaccaacatgagccagctgaccactctgatcctcagctacaatgccctgcagtgcatcctgcctttggccttccagggactccgctccctgcgcctgctgtctctccacggcaatgacatctccaccctccaagagggcatctttgcagacgtgacctccctgtctcacctggccattggtgccaaccccctatactgtgactgccacctccgctggctgtccagctgggtgaagactggctacaaggaaccgggcattgctcgttgtgctgggccccaggacatggagggcaagctgctcctcaccacgcctgccaagaagtttgaatgccaaggtcctccaacgctggctgtccaggccaagtgtgatctctaaatgaatag

SEQ ID NO: 27 - protein, 1122 aa, Homo sapien
MALTPGWGSSAGPVRPELWLLLWAAAWRLGASACPALCTCTGTTVDCHGTGLQAIPKNIPRNTERLELNGNNITRIHKNDFAGLKQLRVLQLMENQIGAVERGAFDDMKELERLRLNRNQLHMLPELLFQNNQALSRLDLSENAIQAIPRKAFRGATDLKNLQLDKNQISCIEEGAFRALRGLEVLTLNNNNITTIPVSSFNHMPKLRTFRLHSNHLFCDCHLAWLSQWLRQRPTIGLFTQCSGPASLRGLNVAEVQKSEFSCSGQGEAGRVPTCTLSSGSCPAMCTCSNGIVDCRGKGLTAIPANLPETMTEIRLELNGIKSIPPGAFSPYRKLRRIDLSNNQIAEIAPDAFQGLRSLNSLVLYGNKITDLPRGVFGGLYTLQLLLLNANKINCIRPDAFQDLQNLSLLSLYDNKIQSLAKGTFTSLRAIQTLHLAQNPFICDCNLKWLADFLRTNPIETSGARCASPRRLANKRIGQIKSKKFRCSAKEQYFIPGTEDYQLNSECNSDVVCPHKCRCEANVVECSSLKLTKIPERIPQSTAELRLNNNEISILEATGMFKKLTHLKKINLSNNKVSEIEDGAFEGAASVSELHLTANQLESIRSGMFRGLDGLRTLMLRNNRISCIHNDSFTGLRNVRLLSLYDNQITTVSPGAFDTLQSLSTLNLLANPFNCNCQLAWLGGWLRKRKIVTGNPRCQNPDFLRQIPLQDVAFPDFRCEEGQEEGGCLPRPQCPQECACLDTVVRCSNKHLRALPKGIPKNVTELYLDGNQFTLVPGQLSTFKYLQLVDLSNNKISSLSNSSFTNMSQLTTLILSYNALQCILPLAFQGLRSLRLLSLHGNDISTLQEGIFADVTSLSHLAIGANPLYCDCHLRWLSSWVKTGYKEPGIARCAGPQDMEGKLLLTTPAKKFECQGPPTLAVQAKCDLCLSSPCQNQGTCHNDPLEVYRCACPSGYKGRDCEVSLDSCSSGPCENGGTCHAQEGEDAPFTCSCPTGFEGPTCGVNTDDCVDHACANGGVCVDGVGNYTCQCPLQYEGKACEQLVDLCSPDLNPCQHEAQCVGTPDGPRCECMPGYAGDNCSENQDDCRDHRCQNGAQCMDEVNSYSCLCAEGYSGQLCEIPP

SEQ ID NO: 28 - protein, 928 aa, Homo sapien
MALTPGWGSSAGPVRPELWLLLWAAAWRLGASACPALCTCTGTTVDCHGTGLQAIPKNIPRNTERLELNGNNITRIHKNDFAGLKQLRVLQLMENQIGAVERGAFDDMKELERLRLNRNQLHMLPELLFQNNQALSRLDLSENAIQAIPRKAFRGATDLKNLQLDKNQISCIEEGAFRALRGLEVLTLNNNNITTIPVSSFNHMPKLRTFRLHSNHLFCDCHLAWLSQWLRQRPTIGLFTQCSGPASLRGLNVAEVQKSEFSCSGQGEAGRVPTCTLSSGSCPAMCTCSNGIVDCRGKGLTAIPANLPETMTEIRLELNGIKSIPPGAFSPYRKLRRIDLSNNQIAEIAPDAFQGLRSLNSLVLYGNKITDLPRGVFGGLYTLQLLLLNANKINCIRPDAFQDLQNLSLLSLYDNKIQSLAKGTFTSLRAIQTLHLAQNPFICDCNLKWLADFLRTNPIETSGARCASPRRLANKRIGQIKSKKFRCSAKEQYFIPGTEDYQLNSECNSDVVCPHKCRCEANVVECSSLKLTKIPERIPQSTAELRLNNNEISILEATGMFKKLTHLKKINLSNNKVSEIEDGAFEGAASVSELHLTANQLESIRSGMFRGLDGLRTLMLRNNRISCIHNDSFTGLRNVRLLSLYDNQITTVSPGAFDTLQSLSTLNLLANPFNCNCQLAWLGGWLRKRKIVTGNPRCQNPDFLRQIPLQDVAFPDFRCEEGQEEGGCLPRPQCPQECACLDTVVRCSNKHLRALPKGIPKNVTELYLDGNQFTLVPGQLSTFKYLQLVDLSNNKISSLSNSSFTNMSQLTTLILSYNALQCILPLAFQGLRSLRLLSLHGNDISTLQEGIFADVTSLSHLAIGANPLYCDCHLRWLSSWVKTGYKEPGIARCAGPQDMEGKLLLTTPAKKFECQGPPTLAVQAKCDL

(19) United States
(12) Patent Application Publication
(10) Pub. No.: US 2004/0109856 A1
(43) Pub. Date: Jun. 10, 2004

(54) Slit1 and MEGF4 isoforms and their use

(76) Inventor: Detlev Mennerich, Berlin (DE)

Correspondence Address: Mayer Fortkort and Williams, 251 North Avenue West, 2nd Floor, Westfield, NJ, 07090, US

(21) Appl. No.: 10/442,658

(22) Filed: May 21, 2003

(30) Foreign Application Priority Data
May 21, 2002 (DE) 102 23 246.6

(51) Int. Cl. A61K 39/395; C07K 14/705

(52) U.S. Cl. 424/143.1; 530/350; 530/388.22

(57) ABSTRACT

The invention relates to new Slit1 or MEGF4 sequences and their uses in connection with the diagnosis and/or treatment of cancer or the screening for substances binding to Slit1 and/or MEGF4, in particular prospective inhibitors.

### FIELD OF THE INVENTION

The invention relates to new Slit1 and MEGF4 isoforms, to the use of sequences derived from Slit1 and MEGF4 for screening for substances binding thereto, and to the use of substances binding to Slit1 and/or MEGF4 for diagnosing and/or treating cancer diseases.

### BACKGROUND OF THE INVENTION AND PRIOR ART

Secreted Slit1 and MEGF4 proteins being different transcriptional isoforms have been well researched in neurological correlations. In the embryogenesis of drosophila, the slit gene plays an essential role for the structure of the central nervous system.

From the document Itoh et al., Molec. Brain Res., 62:175-186 (1998), it is known in the art that three human homologs, Slit1, Slit2 and Slit3, to the slit gene of the drosophila exist. Each of these genes codes a protein comprising conserved protein-protein interactions, for instance leucine-rich repeats (LRR1-LRR4) and EGF-like motives (EGF1-EGF9), similar to the drosophila gene. Human Slit1 is known under XM_{—}005958 or NM_{—}003061. Analyses have shown that a larger 8.4 kb and a smaller 5.9 kb transcript of Slit1 is expressed substantially in the brain. Slit2 and Slit3 are expressed substantially in the spinal cord and the thyroid gland.

According to the document M. Nakajama et al., Genomics, 51:27-34 (1998), the Slit1 gene (there called MEGF4) is on chromosome 10q23.3-q24.

Different documents deal with the role of Slit or MEGF in the generation of and function in neural tissues of mammals. As an example only, reference is made to W. Wu et al., Nature 400:331-336 (1999), W. Yuan et al., Developmental Biology 212:290-306 (1999), T. Kidd et al., Cell 96: 785-794 (1999), Y. Liang et al., JBC 274:17885-17892 (1999), and E. Stein et al., Science, 291: (2001).

In the document JP11164690, the diagnosis and the treatment of cancer diseases is described in the most general form, without any detailed specification of the kind of the cancer disease, by means of a biologically active form of the Slit1 protein.

Cancer, in particular prostate or breast cancer, but also other cancer diseases, is a disease occurring with increasing age at a considerable incidence. Up to now, cancer is diagnosed substantially pathologically and is treated in most cases by removal of the respective organ. The removal of an organ has various disadvantageous effects on a patient. An improved diagnosis and treatment of cancer, in particular without the necessity of removing an organ or of a part thereof, is therefore highly desirable.

### TECHNICAL OBJECT OF THE INVENTION

The invention is based on the technical object to specify pharmaceutical compositions for diagnosing and/or treating cancer diseases.

### BASICS OF THE INVENTION AND PREFERRED EMBODIMENTS

For achieving the technical object, the invention teaches firstly a nucleic acid coding for a Slit1 or MEGF4 isoform comprising a nucleic acid sequence according to Seq.-ID 2, 3, 4, 5, 25, or 26 and a Slit1 or MEGF4 peptide or protein comprising an amino acid sequence according to Seq.-ID 7, 8, 9, 10, 11, 12, 13, 14, 27, or 28. The nucleic acid or the peptide or protein may in particular be composed of these sequences. The said sequences are new isoforms of the human Slit1 or MEGF4. Nucleic acids or proteins or peptides according to the invention can be prepared by the usual molecular biological methods.

The invention further relates to various uses of the new nucleic acids or peptides or proteins, and to (identical) uses of already known Slit1 or MEGF4 nucleic acids. These are:

i) the use of a nucleic acid coding for Slit1 or MEGF4 and/or of a Slit1 or MEGF4 peptide or protein for the detection of cancer or for the detection of a risk of a cancer disease, a tissue sample (of the respective organ, an arbitrary tissue sample for the detection of metastases from the below cancer types) being examined for the transcription or over-transcription of Slit1 or MEGF4 RNA or for the transcription or over-expression of a Slit1 or MEGF4 protein, preferably a nucleic acid coding for Slit1 or MEGF4 or a detector substance binding to a Slit1 or MEGF4 protein or peptide being used, said detector substance preferably comprising a reporter group, the binding of the said nucleic acid and/or the said protein or peptide to the detector substance being detected in a semi-quantitative or quantitative manner, and the cancer disease preferably being selected from the group comprising “prostate cancer, breast cancer, liver cancer, ovarian cancer, colon cancer, pancreas cancer and lung cancer”,

ii) the use of a Slit1 or MEGF4 RNA or of a Slit1 or MEGF4 protein or peptide for screening for substances binding thereto, in particular prospective drugs for inhibiting the said RNA or the said protein or peptide or prospective detector substance, a prospective substance or a mixture of such prospective substances being contacted with the said RNA or the said protein or peptide, binding events being determined by means of a binding assay, and a binding prospective substance being selected, if applicable after deconvolution,

iii) the use of a substance inhibiting Slit1 or MEGF4 for preparing a pharmaceutical composition for the treatment of cancer and/or a metastases generation from a primary tumor, the cancer disease or the primary tumor, respectively, being selected from the group comprising “prostate cancer, breast cancer, liver cancer, ovarian cancer, colon cancer, pancreas cancer and lung cancer”.

A substance used for the invention may be selected from the group comprising:

a) antisense oligonucleotides, siRNA, and ribozymes against a nucleic acid according to claim 1,

b) an organic molecule binding to a peptide or protein according to claim 2, in particular identified according to claim 5, and having a molecular weight below 5,000, preferably below 1,000, most preferably below 300,

c) an aptamer against a protein or peptide according to claim 2, in particular identified according to claim 5,

d) a (monoclonal) antibody, in particular human or humanized antibody against a protein or peptide according to claim 2,

e) an anti-idiotypic non-human (monoclonal) antibody, generated by means of an antibody of the sub-group d), and

f) the above substances derivatized with a reporter group, a cell toxin, an immuno-stimulating component and/or a radio isotope.

In the case a), a hammerhead ribozyme may for instance be used. The ribozyme interface is selected such that by the activity of the ribozyme the expression of the protein is either suppressed, or an inactive form or an inactive fragment of the protein is expressed. Both may for instance be detected by that in a cell system, wherein a protein according to the invention is expressed on a defined level, this cell system is contacted with one or more ribozymes modeled for defined interfaces, and the expression level or the biological activity of the expressed protein is determined. This is then compared to a negative sample or to the results without contacting, and those ribozymes are selected that lead to a lower expression or activity. In a corresponding manner, siRNA or the antisense nucleic acids may be treated. In the case a), all sequences are usable.

In the case b), chemical substance libraries may be employed in order to screen for binding substances. A validation of binding substances for therapeutic purposes may be performed by determination of the biological activity of the protein in a cell system with and without contacting and comparison of the obtained results. For therapeutic purposes, then such substances are selected which lead to a reduced biological activity. It is also possible that in a screening method according to the invention, the biological activity is determined at the position of binding; then a validation in the above sense has been performed together with the screening. Biological activity may for instance be determined by that natural association partners of the protein are determined and the occurrence and form thereof (e.g. monomer/dimer/heterodimer) are examined. Substances generated further downstream in a metabolism cascade may also be used as an indicator; these substances may for instance be identified by that before cell components are analyzed for the cell expressing the protein, and a comparison is made with identical cells, wherein the expression is however gene-technologically deleted. Mimicry compounds being able to specifically bind the functionally active center of the enzyme domains containing proteins and to inhibit them in their biological activity may also be derived. If a mimicry compound is able to specifically bind the peptides, without inhibiting it in its biological function, it may nevertheless contribute to the achievement of the technical and medical object. As a good binder, it may be derivatized with a cell toxin, in order to thus kill the target cells in a targeting approach. Such a cell toxin my be a classic chemotherapeutical, but also a radio isotope. Further, such peptide specific mimicry compounds, derivatized with a reporter group, may be used as a compound in tumor diagnostics.

Suitable aptamers c) may easily be identified for instance by the well known SELEX method, the protein according to the invention being used as a target. All sequences may be employed.

Antibodies d), in particular monoclonal antibodies, may be obtained in a usual way by immunization of a non-human mammal with a protein according to the invention, a nucleic acid according to the invention (e.g. cDNA), a cell (cancer cell or for instance cell transfected with a nucleic acid according to the invention, such as COS or NIH3T3) constitutively expressing a protein according to the invention, or by means of recombinantly prepared protein or peptide, for instance expressed in E. coli or eukaryote cells (e.g. insect cells). Monoclonal antibodies are obtainable by usual selection and establishment of hybridoma cells. The phage display technique may also be used for the generation of the antibodies. For the case d), the following is to be noted. Starting from a sequence according to the invention, an antibody may be derived in a human and/or humanized form, said antibody being able to specifically bind to the functionally active center of the proteins and to inhibit their biological activity. Further, such a peptide specific antibody, derivatized with a reporter group, may be used as a compound in the tumor diagnostics.

In the case of the anti-idiotypic antibodies e), these are obtainable by that by means of an antibody according to the invention that needs not necessarily influence the biological activity of the protein according to the invention, a second anti-idiotypic (monoclonal) antibody is generated in a non-human mammal. This anti-idiotypic antibody feigns then when applied in human cells a picture of the target molecule to the human immune system and is detected as a non-self epitope due to its non-humanized from. Man consequently naturally generates antibodies against the anti-idiotypic antibody and thus also against the protein or against cells expressing the protein. This variant of the invention is exclusively usable for therapeutic purposes. Starting from sequences according to the invention, monoclonal mouse/rat antibodies can be derived. The monoclonal antibodies Ab1 need not necessarily be able to specifically bind the functionally active center of the proteins and inhibit their biological activity. The monoclonal antibodies Ab1 are used in a second step in order to generate second anti-idiotypic mouse monoclonal antibodies aAB1. The monoclonal aAB1's represent the link for the solution of the technical and medical object by “feigning” an image of the antigen to the human immune system and detecting it by its non-humanized form as a non-self epitope. The human body inevitably generates own antibodies against aAB1 and thus against the tumor cells expressing the target protein. Whereas the aAB1 antibodies are exclusively suitable for the treatment of the tumor disease, the Ab1 antibodies are exclusively used for the diagnostic solution of the problem.

In the case f), starting from nucleic acids according to the invention and/or the amino acid sequences, antibodies and/or mimicry compounds can be isolated with a high specific binding activity. Such substances are then in a second step derivatized with cell toxins and used for targeting the tumor cells.

The invention further relates to a method for diagnosing a cancer disease, wherein a detector substance according to the invention in the embodiment with a reporter group is applied into tissue to be examined (being left in the organism or taken therefrom), the tissue to be examined being then subjected to a detection method step being sensitive for the reporter group, and wherein in the case of the detection of a defined minimum value of the reporter group in the tissue the tissue is qualified as containing tumor cells, and to a method for treating a cancer disease, preferably a cancer disease selected from the group comprising “prostate cancer, breast cancer, liver cancer, ovarian cancer, colon cancer, pancreas cancer and lung cancer”, wherein a pharmaceutical composition according to the invention is administered in a physiologically effective dose and usual galenic preparation to a patient.

The invention is based on the finding that Slit1 or MEGF4 is differentially expressed in tumor tissue, in particular prostate or breast tumor tissue, i.e. in these tissues the expression is higher compared to normal cells of the same tissue. This allows on the one hand to use Slit1 or MEGF4 as markers for the identification of tumor cells in suspicious tissue, for instance in the prostate or the breast. On the other hand, the inhibition of Slit1 or MEGF4, in particular also with local application, offers the possibility to influence the tumor-specific Slit1 or MEGF4 associations by other processes in the tumor cells and thus at last to disturb the tumor-specifically modified metabolism and contribute to the death or at least to a growth inhibition of the tumor cells or inhibit the metastasization. Corresponding considerations apply to the case of the metastasization, since Slit1 or MEGF4 is able to inhibit the SDF-1alpha induced, CXCR4 mediated chemotaxis.

For the purpose of the invention, it may be recommendable, prior to a treatment with a pharmaceutical composition according to the invention, to take a sample from a tissue identified by other methods as a tumor tissue, and to examine the tissue sample for expression or over-expression of Slit1 or MEGF4. Alternatively, a test for Slit1 or MEGF4 dependence can be performed with a detector substance according to the invention for in vivo diagnosis. If an expression or over-expression of Slit1 or MEGF4 compared to normal tissue of the same type is detected, the application of the pharmaceutical composition according to the invention is indicated.

If the tumor is a type, wherein tumor cells express Slit1 or MEGF4, not however or only weakly normal cells of the same tissue type, it is particularly preferred that the substance binding to Slit1 or MEGF4 in addition carries a cytotoxic and/or immunostimulating component. This at last leads to that virtually exclusively tumor cells are killed, by cytotoxicity or by an attack by the stimulated immune system, whereas normal cells in the tissue are virtually completely preserved. In this embodiment, the binding substance needs not act inhibitingly on Slit1 or MEGF4, since this binding substance needs only function as a marker carrying the components to target tumor cells. In the case of using a cytotoxic and/or immunostimulating component, it may be particularly recommendable that the pharmaceutical composition is suited for a local application in a tissue containing tumor cells, for instance by injection.

By means of substances according to the invention, in particular peptides and proteins, it is at last possible to perform an immunotherapy by way of vaccination with epitopes of the Slit1.

Definitions.

In this specification, the term Slit1 or

MEGF4 is used for all human isoforms, known or new, on nucleic acid or amino acid basis. Also included in this term are the short sequences disclosed in this specification, said short sequences originating from the isoforms, for instance immunization sequences. Further included are homologs, the homology being at least 80%, preferably more than 90%, most preferably more than 95%. In the case of the nucleic acid sequences, complementary or allelic variants are also included. Further such sequences are included, which represent only partial sequences of the explicitly disclosed sequences, for instance an exon or several exons, or complementary sequences hereto, such that these partial sequences in the case of the nucleic acids comprise a length sufficient for a hybridization with a nucleic acid according to the invention, at least 50 bases, and in the case of the proteins or peptides bind with at least identical affinity to a protein or peptide-specific target molecule. Further, all nucleic acids hybridizing with nucleic acids according to the invention are included, which hybridize under stringent conditions (e.g. 5° C. to 25° C. below the melting temperature; see also J. M. Sambrock et al., A laboratory manual, Cold Spring Harbor Laboratory Press (1989) and E. M. Southern, J Mol Biol, 98:503ff (1975)). It is understood that the invention also covers expression cassettes, i.e. one or more of the nucleic acid sequences according to the invention having at least one control or regulatory sequence. Such an expression cassette may also comprise a sequence for a known protein, in the course of the translation a fusion protein from a known protein and a protein or peptide according to the invention being generated. Further, antisense sequences to the above nucleic acid sequences are also included. Finally, RNA and DNA correlating therewith and vice versa are included, same as genomic DNA as well as correlated cDNA and vice versa.

For the uses according to the invention, the terms Slit1/MEGF4 nucleic acids or proteins or peptides also include, in addition to the full lengths of the disclosed sequences (see also previous paragraph), partial sequences hereof, with a minimum length of 12 nucleotides, preferably 30 to 90 nucleotides in the case of the nucleic acids, and a minimum length of 4 amino acids, preferably 10 to 30 amino acids in case of the peptides or proteins.

The terms of the detection and/or treatment of cancer, for instance prostate cancer and/or breast cancer, also include the detection and/or treatment of metastases from primary tumors in other tissues. The term of the treatment also includes the prophylaxis.

An inhibitor is a compound or substance that either inhibits the generation of Slit1 or MEGF4 or reduces the activity of generated Slit1 or MEGF4, compared to the Slit1 or MEGF4 activity in absence of the inhibitor. In so far an inhibitor may on the one hand be a substance influencing in an inhibiting manner the generation cascade of Slit1 or MEGF4. On the other hand an inhibitor may be a substance that enters in a binding relation with the generated Slit1 or MEGF4, in such a way that further physiological interactions with endogenic substances are at least reduced.

Mimicry compounds are compounds reproducing the variable section, in particular the binding section of an antibody, and bind at the same position of a target molecule as the respective antibody.

The term of the antibodies includes polyclonal antibodies, monoclonal antibodies, non-human, human and humanized antibodies, anti-idiotypic antibodies and phage display antibodies, but also chimeric antibodies and specific fragments of the light and/or heavy chain of the variable section of respective antibodies of the above type. The preparation or recovery of such antibodies with given immunogens is well known to the average man skilled in the art and needs not be explained in more detail here. Further the term of the antibodies includes bispecific antibodies. Bispecific antibodies combine a defined immune cell activity with a specific tumor cell detection, thus tumor cells being killed. A bispecific antibody binds on the one hand to a trigger molecule of the immune effector cell (e.g. CD3, CD16, CD64) and on the other hand to antigens of the tumor target cell.

The galenic preparation of a pharmaceutical composition according to the invention may be performed in a manner suitable for this technology. As counter-ions for ionic compounds may for instance be used Na⁺, K⁺, Li⁺ or cyclohexylammonium. Suitable solid or liquid galenic preparation types are for instance granulates, powders, dragees, tablets, (micro-) capsules, suppositories, syrups, juices, suspensions, emulsions, drops or injectable solutions (IV, IP, IM) and preparations with protractable drug release, for the production of which usual auxiliary means are used, such as carrier substances, blasting, binding, coating, swelling, sliding or lubricating agents, flavors, sweeteners and emulgators. As auxiliary substances are named here magnesium carbonate, titanium dioxide, lactose, mannite and other sugars, talcum, milk protein, gelatin, cellulose and its derivatives, animal and vegetable oils such as cod-liver oil, sunflower, peanut or sesame oil, polyethylene glycols and solvents, such as sterile water and mono or poly-valent alcohols, for instance glycerin. A pharmaceutical composition according to the invention can be prepared by that at least one Slit1/MEGF4 inhibitor is mixed in a defined dose with a pharmaceutically suitable and physiologically carrier and if applicable further suitable drug, additional or auxiliary substances having a defined inhibitor dose and provided in the desired administration manner.

Tumor cells express Slit1 or MEGF4 in a differential manner, whereas normal cells of the same tissue do not express this. Tumor cells over-express Slit1 or MEGF4 in a specific or differential manner, when Slit1 or MEGF4 is, compared to normal cells of the same tissue, expressed higher, for instance at least in a double amount.

Cytotoxic components or groups are compounds directly or indirectly initiating apoptosis or leading to necrosis or at least acting in a growth inhibiting manner. Such groups or compositions may be, in addition to radioisotopes (for instance 188Re, 213Bi, 99 mTc, 90Y, 131J, 177Lu), in particular cytostatic substances used in tumor therapy. Examples for these are alkylating agents (for instance mechlorethamine, ifosfamide, chlorambucil, cyclophosphamide, melphalan, alkyl sulfonates, busulfan, nitrosoureas, carmustine, lomustine, semustine, triazine, dacarbazine), antimetabolites (for instance fol acid antagonists, methotrexate, pyrimidine analogs, fluoruracil, fluordesoxyuridin, cytabarine, gemcitabine, purine analogs, mercaptopurine), mitosis inhibitors (for instance vinca alcaloids, vincristine, vinblastine, paclitaxel, docetaxel, proteaxel), epipodophyl lotoxins (for instance etoposide, teniposide), antibiotics (for instance dactinomycin, daunorubicin, idarubicin, anthracyclines, bleomycin, L-asparaginase), platinum complex compositions (for instance cisplatin), hormones and related compositions (for instance suprarenal gland steroids, aminogluthetimide, gestagens, estrogens, androgens, antiestrogens, tamoxifen, steroid analogs, flutamide). When such a composition is bound to a substance binding Slit1 or MEGF4, the coupling takes place such that the affinity to Slit1 or MEGF4 is reduced by not more than 90%, preferably 50%, referred to the substance without cytostatic group, and the cytostatic effect of the group is reduced by not more than 90%, preferably 50%, referred to the composition without substance.

An immunostimulating component is in most cases a protein or an effective component thereof stimulating cells of the immune system. Examples are cytokines such as M-CSF, GM-CSF, GCSF, interferons such as IFN-alpha, beta, gamma, interleukins such as IL-1 to -16 (except -8), human LIF, chemokines such as rantes, MCAF, MIP-1-alpha, -beta, NAP-1 and IL-8.

A reporter group is an atom, a molecule or a compound permitting in conjunction with a suitably adapted assay the detection of the reporter group and thus of the compound or substance connected with the reporter group. Examples for reporter groups and detection methods associated herewith are: 32P labeling and intensity measurement by using a phosphoimager. Many other examples, for instance detection of fluorescence, are well known to the average man skilled in the art and need not be described in detail.

A substance binding to Slit1 or MEGF4 may be a substance binding a Slit1 or MEGF4 protein or a Slit1 or MEGF4 RNA.

Any terms in the above definition extended in their meaning relative to the precise meaning of the word shall also include the respective terms in the precise meaning of the word.

### EXAMPLES

In the following, the invention is described in more detail, based on examples representing preferred embodiments only. There are:

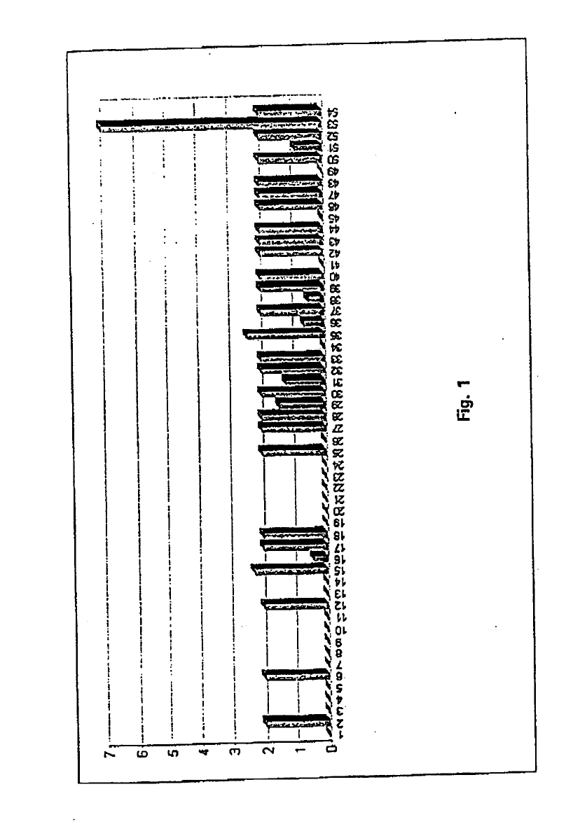

FIG. 1 a chip analysis for the differential expression of Slit1 or MEGF4 in prostate tumor tissues from 54 patients,

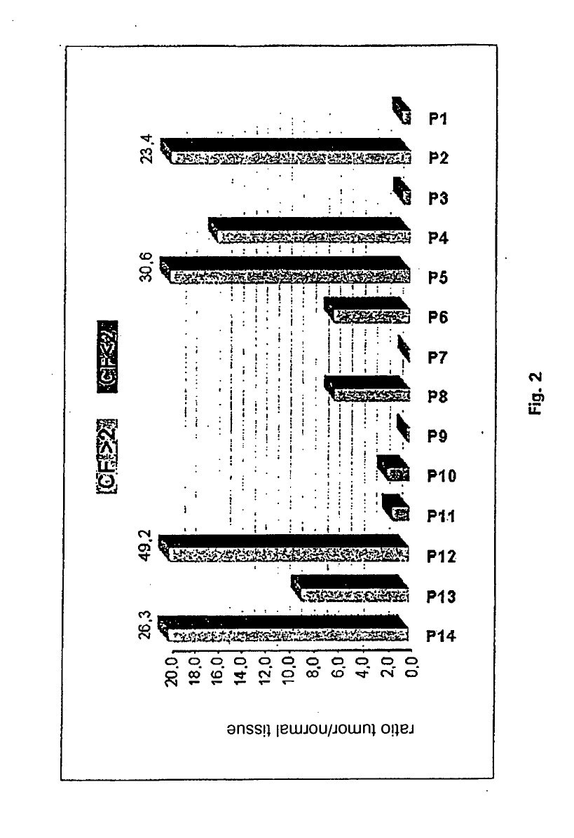

FIG. 2 a Taqman® analysis for the differential expression of Slit1 or MEGF4 in prostate tumor tissues from 14 patients,

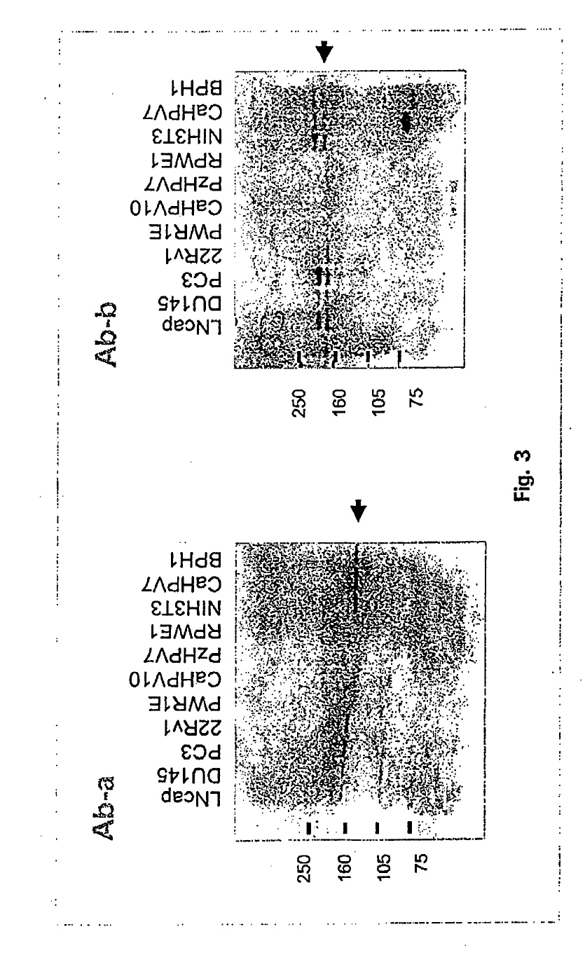

FIG. 3 Western blots for Slit1 or MEGF4 detected by means of antisera according to the invention,

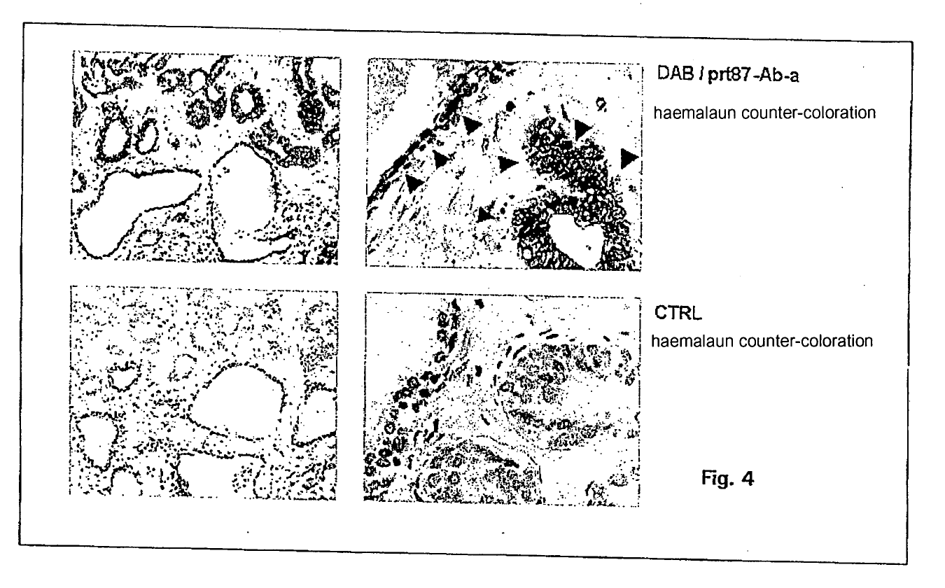

FIG. 4 an IHC detection of Slit1 over-expression in prostate tumor cells,

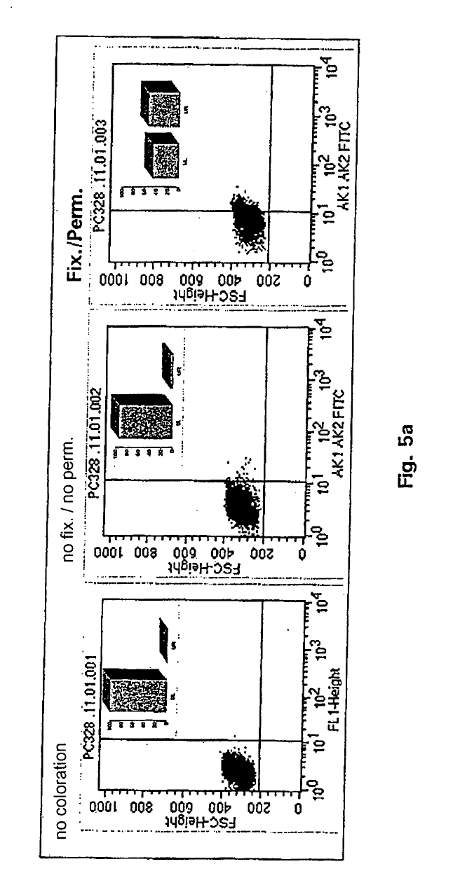

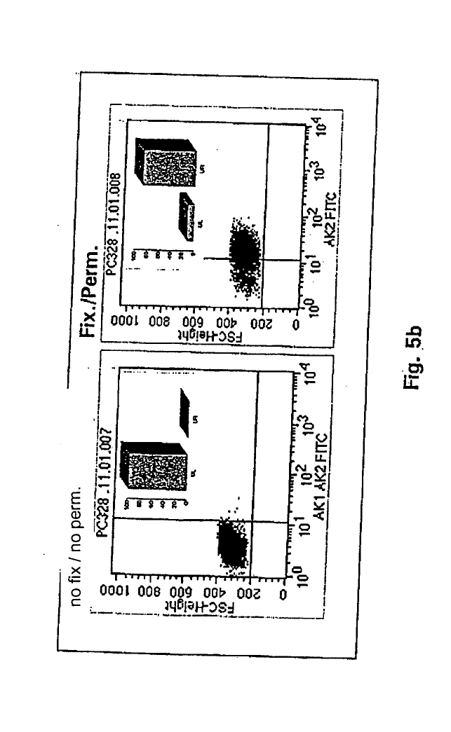

FIG. 5 a Facs analysis for the localization of the Slit1 or MEGF4 protein,

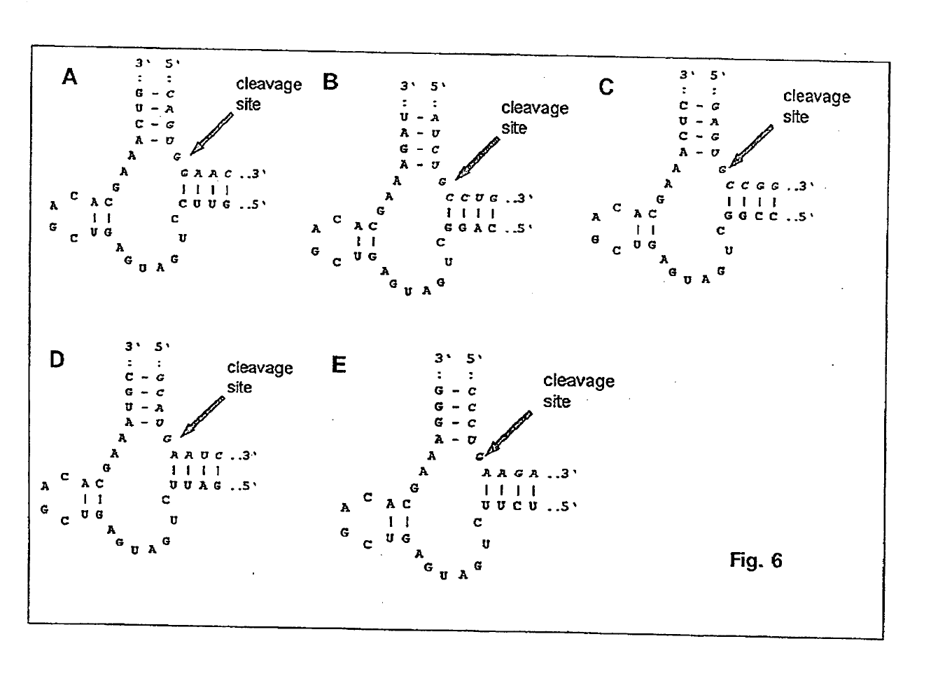

FIG. 6 5 different hammerhead ribozymes cutting Slit1 or MEGF4 or tSlit1 (Seq.-ID 5) mRNA,

FIG. 7 the detection of recombinant human SLIT from stably transfected HEK293 cells with the antibody prt87 B2,

FIG. 8 the detection of the binding of SLIT1 to heparin agarose,

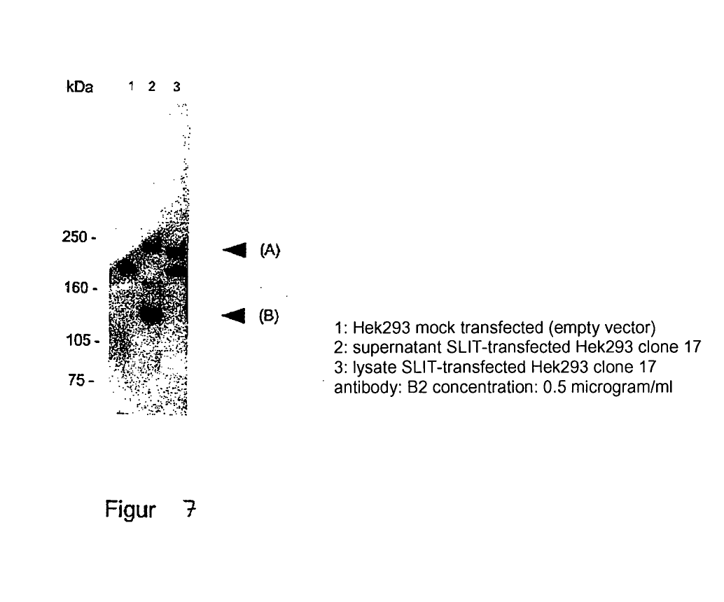

FIGS. 9 to 14 the purification of SLIT1,

FIG. 15 a graphic representation of shortened peptide sequences including domains.

### Example 1

### Over-Expression in Prostate Tumor

Paired tumor and normal tissue was taken by laser microdissection from 54 patients. The RNA prepared therefrom was labeled with dioxygenin and hybridized on a DNA chip by means of the Affymetrix technology. The results of the analysis of the chip are shown in FIG. 1 (ordinate: expression ratio tumor/normal tissue; abscissa: 54 patients having prostate tumor). It can be seen that in 26 of 54 patients there is no differential expression, in 2 of 54 there is a down-regulation and in 26 of 54 there is an up-regulation of Slit1/MEGF4 (factor >2).

This finding was checked by means of the Taqman® analysis. Paired tumor and normal tissue of 14 patients was obtained by laser microdissection. The RNA prepared therefrom was reversely transcribed. The thus obtained cDNA was used as a template in quantitative PCR. The results are shown in FIG. 2 (ordinate same as in FIG. 1; abscissa: 14 patients having prostate tumor). No differential expression of Slit1/MEGF4 was detected in 6 of 14 patients, and an up-regulation of the transcription was detected in 8 of 14.

Thus it is proven that the expression of Slit1/MEGF4 in at least 50% of the prostate tumors are up-regulated, so that Slit1/MEGF4 is suited for the detection of prostate tumors, and that a physiological relation between up-regulation and the disease symptoms and thus an inhibition of Slit1/MEGF4 is suited for the treatment of the tumor disease.

### Example 2

### Detection of Slit1/MEGF4 by Means of Antibodies

Polyclonal antibodies (AB) were grown against Slit1 protein (Seq.-ID 9; AB-a) and against human SLIT1/MEGF4 proteins (Seq.-ID 10; AB-b), conjugated with a carrier protein, in rabbit and affinity purified with the specific immobilized peptides.

With the above AB's, Western blots were made for the detection of Slit1 and/or MEGF4 in prostate tumor and non-malignant normal prostate epithelium cell lines and in breast tumor cell lines. For this purpose, lysates from the whole cells (wc) or of the membrane fraction (mem) were prepared, denaturated and separated by means of SDS phage gel electrophoresis. After blotting the separated proteins on a membrane and blocking unspecific protein interactions, the membrane was incubated with AB-a and/or AB-b antisera over night at 4° C. With an anti-rabbit coupled horse radish peroxidase as a secondary antibody, the specific Slit1 and/or MEGF4 bands were detected. The results are shown in FIG. 3. By means of AB-a was made the detection in all cell lines at a molecular weight of approx. 190 kD. By means of AB-b, Slit1 (approx. 190 kD) as well as MEGF4 (approx. 180 or 210 kD) became visible. The specificity of the detected proteins was verified with not shown competition experiments under addition of specific or unspecific peptides at the incubation with the AB's.

In addition to the expression shown in FIG. 3 of Slit1/MEGF4 protein in prostate tumor cell lines, an expression was found in not shown experiments in the breast tumor cell lines MaTu and MDA 231 or MDA-MB-231.

### Example 3

### Immunohistochemical Detection of Prostate Tumor Cells

Tissue samples from prostate tumor cells were incubated with AB-a as a primary antibody, a biotinylated secondary anti-rabbit antibody and a streptavidin-coupled horse radish peroxidase, and the coloration was performed with DAB as a chromogenic substrate (brown coloration). The counter-coloration took place with a haemalaun solution (blue coloration). Malignant and normal epithelium cells were distinguishable, the malignant cells having a strong coloration, i.e. high Slit1 content, whereas the normal epithelium cells were only moderately colored. The cuts are shown in FIG. 4, same as the negative controls performed without AB-a.

### Example 4

In this example, the localization of Slit1 or MEGF4 was examined. For this purpose, PC3 (prostate tumor cell line) cells were subjected to a Facs analysis under application of AB-a and AB-b for Slit1 and for Slit1/MEGF4.

In FIG. 5a, the results for AB-a and in FIG. 5b the results for AB-b are shown. On the left-hand side, it can be seen that on the cell membrane of living, intact cells no Slit1/MEGF4 can be detected. After the permeabilization however follows a detection of the intracellularly localized proteins (right-hand side).

### Example 5

### RNA Inhibitors

In FIG. 6 are shown various hammerhead ribozymes cutting Slit1 or MEGF4 at the shown positions and thus inhibit the activity of any translation products or at least the quantity thereof. The respective sequences of the figure parts a to e are Seq.-ID 20 to 24.

The sequences Seq.-ID 15 and 16 are antisense sequences for Slit1 as well as for MEGF4 RNA. The sequences Seq.-ID 17, 18 and 19 are antisense sequences for Slit1, MEGF4 and tSlit1, respectively.

### Example 6

### Antibody Detection of Recombinant Slit

HEK293 cells were stably transfected in a conventional way with a Slit nucleic acid. Against the corresponding protein, prt87 B2 antibodies were generated in a conventional way. In FIG. 7 can be seen that Slit can be detected in the supernatant of the cultivated HEK293 cells as well as in the lysate (A). The slight height difference in tracks 2 and 3 can be put down to the different buffer conditions in the lysate or in the supernatant at the electrophoresis. In the supernatant can be detected a small protein of approx. 140 kDa (B), which might correspond to a hypothetical fission product of Slit. The results show that the antibody specifically detects Slit. Slit is secreted in the cell culture supernatant and is cut at the potential fission position.

### Example 7

### Binding of Slit to Heparin Agarose

Supernatant or lysate of cells from example 6 was incubated with heparin agarose. After washing the beads, the whole protein bound to the beads was eluated with a SDS-containing buffer and applied to a gel. Slit was detected by means of the antibody from example 6. From the lysate as well as from the supernatant, the complete Slit1 molecule (A) can be enriched with heparin. The shortened form from the supernatant (B) does not bind to heparin, which is in agreement with the predicted fission position.

### Example 8

### Isolation or Purification of Slit

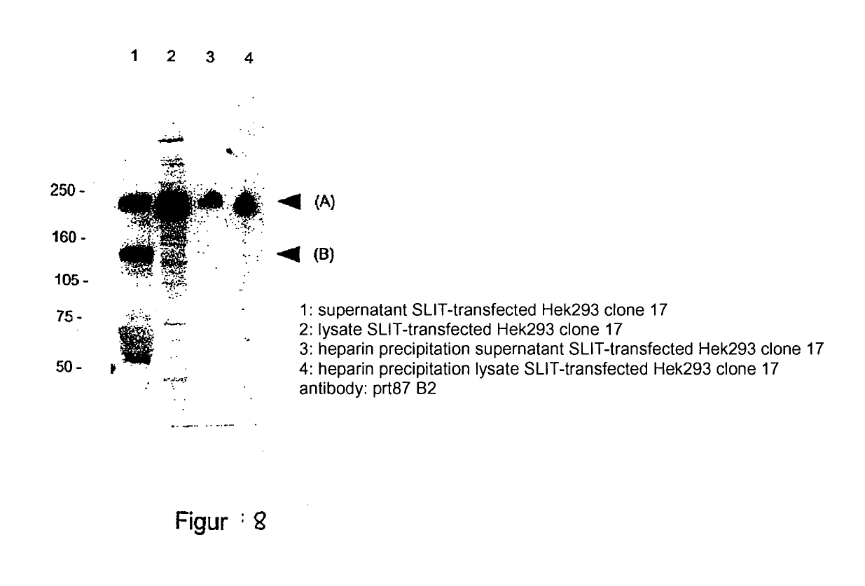

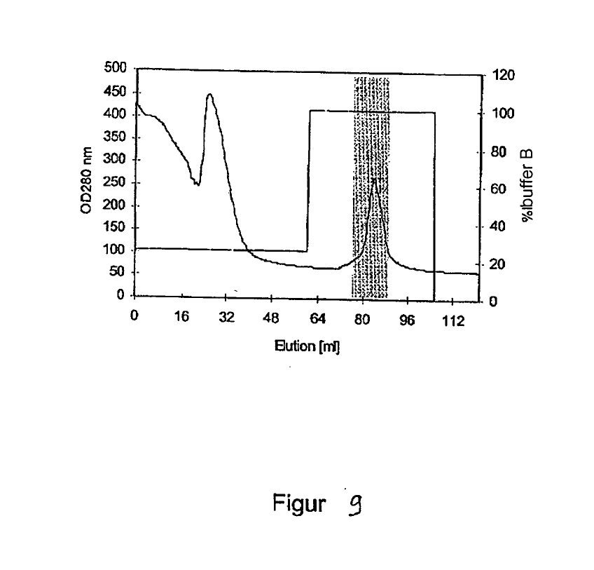

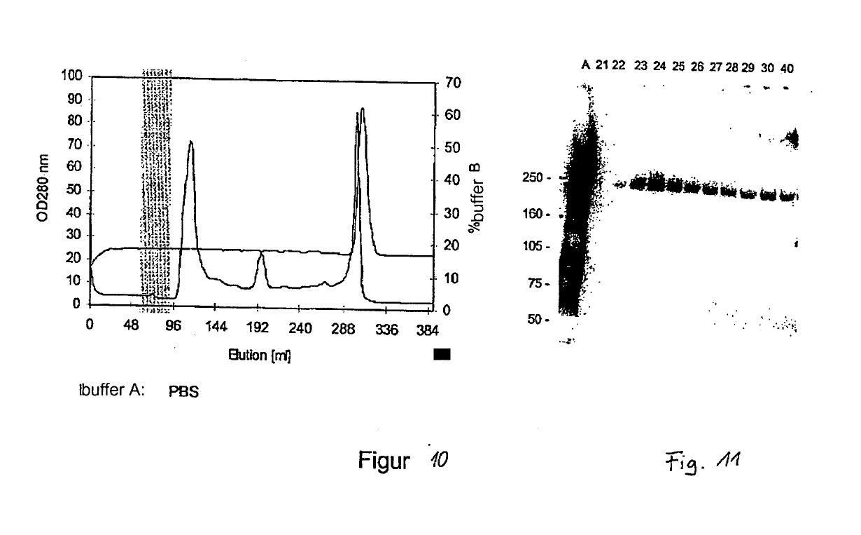

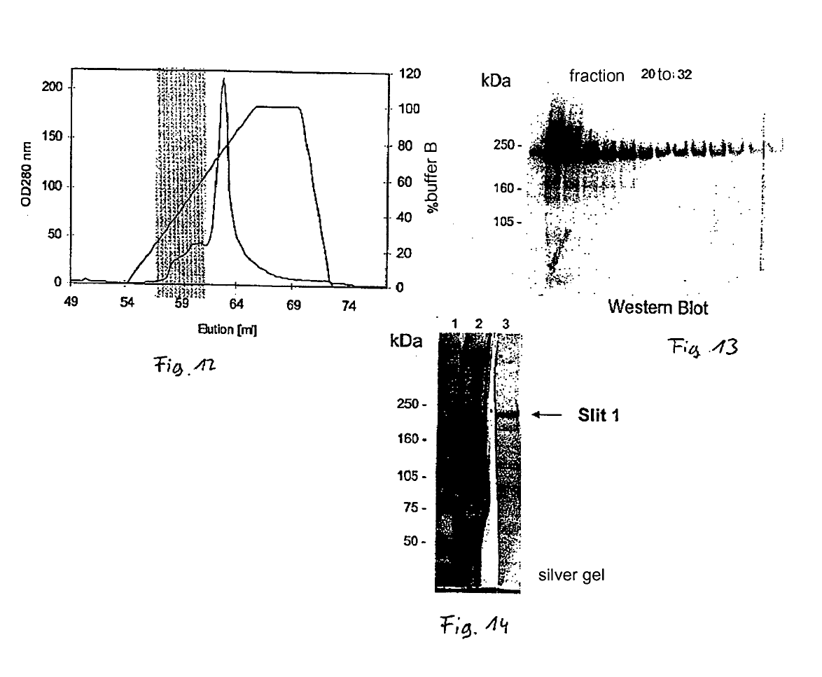

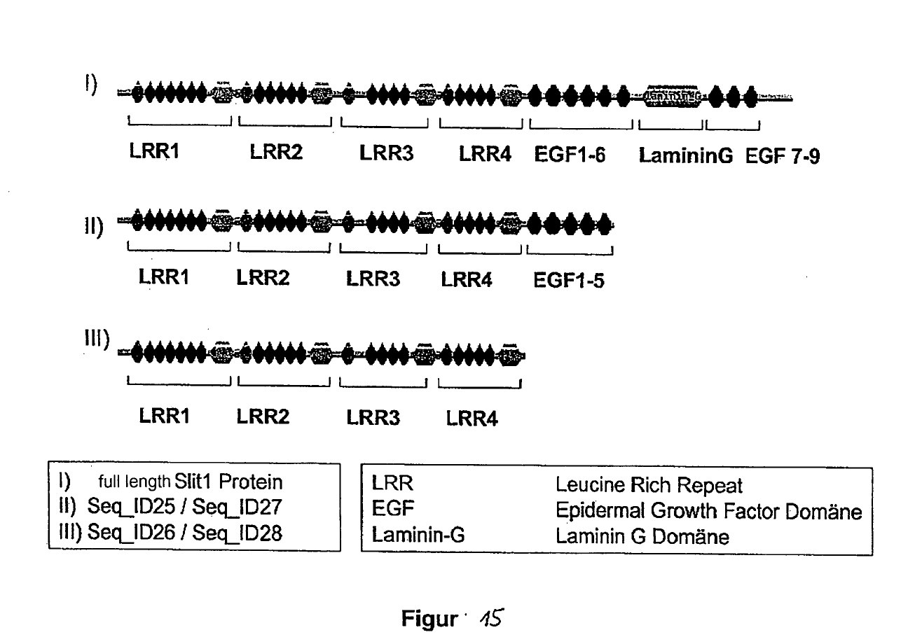

Cells from example 6 were cultivated in DMEM with 10% serum, and 2,000 ml culture supernatant were obtained. This was concentrated over 50 kDa cut-off membrane to 200 ml. Therefrom samples were fed to a 20 ml heparin affinity chromatography column operated with 75% buffer A and 25% buffer B. Slit1 was then eluated with 100% buffer B from the column. The fractions 11-14 (light gray, see FIG. 9) were united and concentrated to 7 ml. The further purification was performed by a gel chromatography column (Superdex200 26/60 column), the elution taking place at 1 ml/min, and reference is made to FIG. 10. The analysis was made with a Western blot with the antibody from example 6 (FIG. 11). The fractions 21 to 28 (see FIG. 10) were united and adjusted to 2 M urea (final volume 50 ml). This sample was used after dialysis directly for QA-CIM-disk (ion exchange chromatography, 350 μl), and reference is made to FIG. 12. The elution was made with an increasing concentration of the buffer B (NaCl gradient). The fractions 20-32 were separated in a Western blot (FIG. 13), and Slit1 was detected with the antibody of example 6. Thereafter the fractions 20-25 containing Slit1 were united and analyzed in a silver gel (FIG. 14). As a result, Slit1 is detected in a purified form and with the molecular weight to be expected.

### Example 9

### Biological Activity of Slit Fragments

In FIG. 15, Pfam representations of the Slit1 full length proteins (I) as well as of 2 N-terminal fragments according to the sequences 25 to 28 (II and III) are shown. LLR means leucine rich repeat. EGF means epidermal growth factor domain. Laminin G means this domain. The fragment II is detectable according to example 6 (band B), and is apparently the result of an endogenic proteolytic fission. It comprises the LRR's 1-4 and the first 5 EGF. Since these protein domains are autonomously folding, also the recombinant fragment II will comprise a normal folding and a complete biological activity. Corresponding considerations apply to the fragment III with the LRR's 1-4 with its biological activity as a chemorepellent.

Legend.

FIG. 2:

Ratio tumor/normal tissue

FIG. 4:

haemalaun counter-coloration

haemalaun counter-coloration

FIG. 5a:

no coloration no fix./no perm. fix/perm.

FIG. 5b:

no fix./no perm. fix/perm.

FIG. 7:

1: Hek293 mock transfected (empty vector)

2: supernatant SLIT-transfected Hek293 clone 17

3: lysate SLIT-transfected Hek293 clone 17 antibody: B2 concentration: 0.5 μg/ml

FIG. 8:

1: supernatant SLIT-transfected Hek293 clone 17

2: lysate SLIT-transfected Hek293 clone 17

3: heparin precipitation supernatant SLIT-transfected Hek293 clone 17

4: heparin precipitation lysate SLIT-transfected Hek293 clone 17

antibody: prt87 B2

FIG. 10

buffer A: PBS % buffer cB

FIG. 13

fractions 20 to 32

FIG. 14

silver gel

FIG. 15

I) full length Slit1 protein

II) Seq_ID25/Seq_ID27

III) Seq_ID26/Seq_ID28

1. A nucleic acid, in particular an isolated nucleic acid, coding for a Slit1 or MEGF4 isoform or a fragment hereof, comprising a nucleic acid sequence with a sequence according to Seq.-ID 2, 3, 4, 5, 25, or 26, or consisting hereof.

2. A Slit1 or MEGF4 peptide or protein, in particular isolated, comprising an amino acid sequence coded by a nucleic acid according to claim 1 or consisting hereof, or according to Seq.-ID 7, 8, 9, 10, 11, 12, 13, 14, 27, or 28 or consisting hereof, the protein or peptide preferably being functional in the generation and function of neural tissues of mammals.

3. The use of a nucleic acid coding for Slit1 or MEGF4 and/or a Slit1 or MEGF4 peptide, in particular according to claim 1 or 2, for the detection of cancer or for the detection of a risk of the disease of cancer, wherein a tissue sample is examined for transcription or over-transcription of Slit1 or MEGF4 RNA or for expression or over-expression or a Slit1 or MEGF4 protein, the cancer disease being selected preferably from the group comprising “prostate cancer, breast cancer, liver cancer, ovarian cancer, colon cancer, pancreas cancer and lung cancer”.

4. The use according to claim 3, wherein a detector substance binding to a nucleic acid coding for Slit1 or MEGF4 or to a Slit1 or MEGF4 protein or peptide, preferably comprising a reporter group, is used, the binding of the said nucleic acid and/or of the said protein or peptide to the detector substance being detected in a semi-quantitative or quantitative manner.

5. The use of a Slit1 or MEGF4 DNA, RNA or of a Slit1 or MEGF4 protein or peptide, in particular according to claim 1 or 2, and/or of a cell expressing such protein or peptide for screening for substances binding thereto, in particular prospective drugs for inhibiting the said DNA or RNA or the said protein or peptide or prospective detector substance, a prospective substance or a mixture of such prospective substances being contacted with the said DNA, RNA or the said protein or peptide, binding events being determined by means of a binding assay, and a binding prospective substance being selected, if applicable after deconvolution.

6. The use of a substance inhibiting Slit1 or MEGF4 or binding thereto, in particular identified according to claim 5, for preparing a pharmaceutical composition for the treatment of cancer and/or a metastases generation from a primary tumor, the cancer disease or the primary tumor, respectively, preferably being selected from the group comprising “prostate cancer, breast cancer, liver cancer, ovarian cancer, colon cancer, pancreas cancer and lung cancer”.

7. The use according to claim 6, wherein the substance is selected from the group comprising:
a) antisense oligonucleotides, siRNA, and ribozymes against a nucleic acid according to claim 1,
b) an organic molecule binding to a peptide or protein according to claim 2, in particular identified according to claim 5, and having a molecular weight below 5,000, preferably below 1,000, most preferably below 300,
c) an aptamer against a protein or peptide according to claim 2, in particular identified according to claim 5,
d) a (monoclonal) antibody, in particular human or humanized antibody against a protein or peptide according to claim 2,
e) an anti-idiotypic non-human (monoclonal) antibody, generated by means of an antibody of the sub-group d), and
f) the above substances derivatized with a reporter group, a cell toxin, an immuno-stimulating component and/or a radio isotope.

8. The use according to claim 7, wherein the substance is a mimicry compound of an antibody against a MEGF4 peptide or protein, in particular according to claim 2.

9. The use according to claim 7, wherein the substance is an aptamer, an antisense RNA according to one of the nucleic acid sequences Seq.-ID 15 to 19, or a ribozyme according to one of the nucleic acid sequences Seq.-ID 20 to 24.

10. The use according to one of claims 6 to 9, wherein the substance in addition carries a cytotoxic and/or immunostimulating component.

11. The use according to one of claims 6 to 10, wherein the pharmaceutical composition is provided for the local application in tissue containing tumor cells.

12. A method for diagnosing a cancer disease, wherein a detector substance according to claims 6 to 11 in the embodiment with a reporter group is applied in vitro or in vivo into tissue to be examined or is contacted with a blood serum sample, the tissue to be examined or the blood serum sample being then subjected to a detection method step being sensitive for the reporter group, and wherein in the case of the detection of a defined minimum value of the reporter group in the tissue, the tissue of a respective patient is qualified as containing tumor cells.

13. A method for treating a cancer disease, preferably a cancer disease, selected from the group comprising “prostate cancer, breast cancer, liver cancer, ovarian cancer, colon cancer, pancreas cancer and lung cancer”, wherein a pharmaceutical composition according to one of claims 6 to 11 is administered in a physiologically effective dose to a patient.

14. A vector containing a nucleic acid according to claim 1.

15. A cell, in particular selected from the group comprising “HEK293, Sf9 and High Five insect cells”, transfected with a nucleic acid according to claim 1 or a vector according to claim 14.
